(19) United States
(12) Patent Application Publication
(10) Pub. No.: US 2023/0381586 A1
(43) Pub. Date: Nov. 30, 2023

(54) SYSTEM AND METHOD FOR IMPROVING AN ATHLETIC BODY POSITION

(71) Applicant: MOTUS DESIGN GROUP LTD., Victoria (CA)

(72) Inventors: Joshua Gregg Erickson, Victoria (CA); Jeffrey Owen Doyle, Victoria (CA)

(21) Appl. No.: 18/030,903

(22) PCT Filed: Oct. 8, 2021
(86) PCT No.: PCT/CA2021/051420

§ 371 Date: Apr. 7, 2023

Related U.S. Application Data
(60) Provisional application No. 63/090,208, filed on Oct. 10, 2020.

(51) Int. Cl. A63B 24/00

(52) U.S. Cl. CPC A63B 24/0062; A63B 24/0075

(57) ABSTRACT

Described are various devices, systems, and methods for determining an improved athletic body position during performance of an activity. One embodiment relates to a system comprising a digital data processor, a user interface, and a computer-readable medium having digital instructions stored thereon. The instructions are executable by the digital processor to access a digital dataset comprising a plurality of respective athletic body positions, each having associated therewith a respective aerodynamic interaction metric and a respective athletic output. The system may further receive as input an environmental interaction parameter, and, based at least in part thereon, compute the optimal athletic body position. The system may further display a signal corresponding to the optimal athletic body position via the user interface.

### CROSS REFERENCE TO RELATED APPLICATIONS

This application claims priority to U.S. Patent Application No. 63/090,208 filed Oct. 10, 2020, the entire disclosure of which is hereby incorporated herein by reference.

### FIELD OF THE DISCLOSURE

The present disclosure relates to athletic monitoring systems and devices, and, in particular, to a system and method for improving an athletic body position.

### BACKGROUND

Athletic performance monitoring systems and devices are commonly used in various athletic disciplines to assist both professional and amateur athletes alike to improve their form, training methods and/or results overall through sensored feedback and data analysis. Equipment testing and development is also routinely subject to these types of analyses.

Cycling is one example of a competitive sport that is increasingly employing sophisticated methods for improving rider performance and bicycle equipment component design. For instance, over the last decade, sensors known as power meters have become increasingly mainstream in the market that directly measure the mechanical power output of a cyclist. As a large fraction of the power that an athlete produces goes into overcoming wind resistance, understanding aerodynamic drag on an athlete and mitigating the effects thereof via techniques such as drafting or minimising a frontal area (and therefore drag) are of particular importance in not only cycling, but may endurance sports.

Ultimately, however, various competing factors contribute to athletic performance. Parameters such as metabolic efficiency, aerodynamics, and the extent to which an athlete can produce or maintain an output power all play a role in determining athletic performance, and can each be affected to different extents by to how an athlete chooses to perform a particular activity.

This background information is provided to reveal information believed by the applicant to be of possible relevance. No admission is necessarily intended, nor should be construed, that any of the preceding information constitutes prior art or forms part of the general common knowledge in the relevant art.

### SUMMARY

The following presents a simplified summary of the general inventive concept(s) described herein to provide a basic understanding of some aspects of the disclosure. This summary is not an extensive overview of the disclosure. It is not intended to restrict key or critical elements of embodiments of the disclosure or to delineate their scope beyond that which is explicitly or implicitly described by the following description and claims.

A need exists for a system and method for improving an athletic body position that overcome some of the drawbacks of known techniques, or at least, provides a useful alternative thereto. Some aspects of this disclosure provide examples of such systems, devices and methods.

In accordance with one aspect, there is provided a computer-readable medium having digital instructions stored thereon and executable by one or more digital processors to automatically determine an optimal athletic body position for an athlete performing an activity, the instructions executable to: access a digital dataset comprising a plurality of respective athletic body positions, each having associated therewith a respective aerodynamic interaction metric and a respective athletic output; receive as input an environmental interaction parameter; compute, at least in part using the environmental interaction parameter and the digital dataset, the optimal athletic body position for a designated athletic output; and output a signal corresponding to the optimal athletic body position.

In one embodiment, the instructions are further executable to: receive as input a current body position metric indicative of a current athletic body position during performance of the activity; and classify, based on the current body position metric, one of the respective athletic body positions as the current athletic body position; wherein the signal corresponding to the optimal athletic body position further comprises information related to the current athletic body position. In one embodiment, the instructions are further executable to perform a comparison of the current athletic body position with the optimal athletic body position, and the signal comprises information related to the comparison.

In one embodiment, the instructions are executable during performance of the activity.

In one embodiment, the instructions are further executable to quantify an athletic output efficiency based at least in part on the current athletic body position and the optimal athletic body position, and wherein the signal further comprises data related to the athletic output efficiency.

In one embodiment, the instructions are further executable to track the athletic output efficiency over a designated duration of the physical activity so to provide an accumulated output efficiency.

In one embodiment, the instructions are further executable to communicate the signal to a third-party digital application using an internet protocol or a wired connection.

In one embodiment, the respective athletic output is related to one or more of an energy output, a power output, a metabolic cost, or a power duration curve.

In one embodiment, the designated athletic output corresponds to one or more of a designated speed, a designated power, a designated efficiency, or a designated athletic output as a function of metabolic cost.

In one embodiment, a designated relationship between the plurality of respective athletic body positions, the respective aerodynamic interaction metrics, and the respective athletic outputs is predetermined from data acquired from prior performance of the athletic activity by multiple athletes.

In one embodiment, a designated relationship between the plurality of respective athletic body positions, the respective aerodynamic interaction metrics, and the respective athletic outputs is predetermined from data acquired from prior performance of the athletic activity the athlete.

In accordance with another aspect, there is provided a system for automatically determining an optimal athletic body position for an athlete performing an activity, the system comprising a digital data processor, a user interface, and a computer-readable medium having digital instructions stored thereon and executable by the digital processor to: access a digital dataset comprising a plurality of respective athletic body positions, each having associated therewith a respective aerodynamic interaction metric and a respective athletic output; receive as input an environmental interaction parameter; compute, at least in part using the environmental interaction parameter and the digital dataset, the optimal athletic body position for a target athletic output; and display via said user interface a signal corresponding to the optimal athletic body position.

In one embodiment, the instructions are further executable to: receive as input a current body position metric indicative of a current athletic body position during performance of the activity; and classify, based on the current body position metric, one of the respective athletic body positions as the current athletic body position; wherein the signal corresponding to the optimal athletic body position further comprises information related to the current athletic body position. In one embodiment, the instructions are further executable to perform a comparison of the current athletic body position with the optimal athletic body position, and the signal comprises information related to the comparison.

In one embodiment, the instructions are executed during performance of the activity and operable to provide real-time feedback related to the athlete.

In one embodiment, the instructions are further executable to quantify an athletic output efficiency based at least in part on the current athletic body position and the optimal athletic body position, and wherein the signal further comprises data related to the athletic output efficiency.

In one embodiment, the instructions are further executable to track the athletic output efficiency over a designated duration of the physical activity so to provide an accumulated output efficiency.

In one embodiment, the instructions are further executable to communicate the signal to a third-party digital application using an internet protocol or a wired connection.

In one embodiment, the respective athletic output is related to one or more of an energy output, a power output, a metabolic cost, or a power duration curve.

In one embodiment, the designated athletic output corresponds to one or more of a designated speed, a designated power, a designated efficiency, or a designated athletic output as a function of metabolic cost.

In one embodiment, a designated relationship between the plurality of respective athletic body positions, the respective aerodynamic interaction metrics, and the respective athletic outputs is predetermined from data acquired from prior performance of the athletic activity by multiple athletes.

In one embodiment, a designated relationship between the plurality of plurality of respective athletic body positions, said respective aerodynamic interaction metrics, and the respective athletic outputs is predetermined from data acquired from prior performance of the athletic activity the athlete.

In accordance with another aspect, there is provided a method of automatically determining an optimal athletic position for an athlete performing an activity, the method to be implemented by one or more digital data processors and comprising: accessing a digital dataset comprising a plurality of respective athletic body positions, each having associated therewith a respective aerodynamic interaction metric and a respective athletic output; receiving as input an environmental interaction parameter; computing, at least in part using the environmental interaction parameter and the digital dataset, the optimal athletic body position for a target athletic output; and outputting a signal corresponding to the optimal athletic body position.

In one embodiment, the method further comprises: receiving as input a current body position metric indicative of a current athletic body position during performance of the activity; and classifying, based on the current body position metric, one of the respective athletic body positions as the current athletic body position; wherein the signal corresponding to the optimal athletic body position further comprises information related to the current athletic body position. In one embodiment, the method further comprises comparing the current athletic body position with the optimal athletic body position, and the signal comprises information related to said comparing.

In one embodiment, the method further comprises displaying the signal in real-time to the athlete.

In one embodiment, the method further comprises quantifying an athletic output efficiency based at least in part on the current athletic body position and the optimal athletic body position, and wherein the signal further comprises data related to the athletic output efficiency.

In one embodiment, the method further comprises tracking the athletic output efficiency over a designated duration of the physical activity so to provide an accumulated output efficiency.

In one embodiment, the method further comprises communicating the signal to a third-party digital application using an internet protocol or a wired connection.

In one embodiment, the method further comprises generating the dataset from data acquired from prior performance of the athletic activity by multiple athletes.

In one embodiment, the method further comprises generating the dataset from data acquired from prior performance of the athletic activity the athlete.

Other aspects, features and/or advantages will become more apparent upon reading of the following non-restrictive description of specific embodiments thereof, given by way of example only with reference to the accompanying drawings.

### BRIEF DESCRIPTION OF THE FIGURES

Several embodiments of the present disclosure will be provided, by way of examples only, with reference to the appended drawings, wherein:

FIG. 1 is a diagram of various forces and parameters involved in an exemplary embodiment of an aerodynamic drag monitoring system and method when applied to cycling;

FIG. 2 is a diagram of an aerodynamic drag monitoring system, in accordance with one embodiment, when illustratively applied to cycling;

FIG. 3 is an illustrative plot of a power-duration curve of a cyclist, in accordance with one embodiment;

FIG. 4 is an illustrative plot of power output by cyclists as a function of torso angle;

FIG. 5 is an illustrative plot of power output by a cyclist as a function of mean hip angle;

FIG. 6 is an illustrative plot of five exemplary power duration curves corresponding to respective cyclist body positions, in accordance with various embodiments;

FIG. 7 is an image of a nacelle operable to measure air speed, in accordance with one embodiment;

FIG. 8A is an image of four exemplary cyclist body positions resulting in different frontal areas, and FIG. 8B is a plot of drag area as a function of cyclist torso angle, in accordance with one embodiment;

FIG. 9 is an illustrative plot of a five 30 second power-duration curves corresponding to respective cyclist body positions, in accordance with various embodiments;

FIG. 10 is a schematic diagram of a user interface for providing body position-related feedback to an athlete, in accordance with various embodiments;

FIGS. 11A to 11D are schematic diagrams of user interface for providing body position-related feedback to an athlete, in accordance with various embodiments;

FIG. 12 is a diagram of a system employing sensor data for real-time optimisation of athletic body position, in accordance with one embodiment, when illustratively applied to cycling;

FIG. 13 is a diagram of a process for determining an improved athletic body position, in accordance with various embodiments;

FIG. 14 is a diagram of an exemplary process for indicating an optimal or improved athletic body position for an athlete performing an activity, in accordance with one embodiment;

FIG. 15 is a a schematic diagram of an aerodynamic drag monitoring system, in accordance with one embodiment; and

FIG. 16 is a schematic diagram of an athlete body position optimization and feedback system, in accordance with one embodiment.

Elements in the several figures are illustrated for simplicity and clarity and have not necessarily been drawn to scale. For example, the dimensions of some of the elements in the figures may be emphasized relative to other elements for facilitating understanding of the various presently disclosed embodiments. Also, common, but well-understood elements that are useful or necessary in commercially feasible embodiments are often not depicted in order to facilitate a less obstructed view of these various embodiments of the present disclosure.

### DETAILED DESCRIPTION

Various implementations and aspects of the specification will be described with reference to details discussed below. The following description and drawings are illustrative of the specification and are not to be construed as limiting the specification. Numerous specific details are described to provide a thorough understanding of various implementations of the present specification. However, in certain instances, well-known or conventional details are not described in order to provide a concise discussion of implementations of the present specification.

Various apparatuses and processes will be described below to provide examples of implementations of the system disclosed herein. No implementation described below limits any claimed implementation and any claimed implementations may cover processes or apparatuses that differ from those described below. The claimed implementations are not limited to apparatuses or processes having all of the features of any one apparatus or process described below or to features common to multiple or all of the apparatuses or processes described below. It is possible that an apparatus or process described below is not an implementation of any claimed subject matter.

Furthermore, numerous specific details are set forth in order to provide a thorough understanding of the implementations described herein. However, it will be understood by those skilled in the relevant arts that the implementations described herein may be practiced without these specific details. In other instances, well-known methods, procedures and components have not been described in detail so as not to obscure the implementations described herein.

In this specification, elements may be described as “configured to” perform one or more functions or “configured for” such functions. In general, an element that is configured to perform or configured for performing a function is enabled to perform the function, or is suitable for performing the function, or is adapted to perform the function, or is operable to perform the function, or is otherwise capable of performing the function.

It is understood that for the purpose of this specification, language of “at least one of X, Y, and Z” and “one or more of X, Y and Z” may be construed as X only, Y only, Z only, or any combination of two or more items X, Y, and Z (e.g., XYZ, XY, YZ, ZZ, and the like). Similar logic may be applied for two or more items in any occurrence of “at least one . . . ” and “one or more . . . ” language.

Unless defined otherwise, all technical and scientific terms used herein have the same meaning as commonly understood by one of ordinary skill in the art to which this invention belongs.

Throughout the specification and claims, the following terms take the meanings explicitly associated herein, unless the context clearly dictates otherwise. The phrase “in one of the embodiments” or “in at least one of the various embodiments” as used herein does not necessarily refer to the same embodiment, though it may. Furthermore, the phrase “in another embodiment” or “in some embodiments” as used herein does not necessarily refer to a different embodiment, although it may. Thus, as described below, various embodiments may be readily combined, without departing from the scope or spirit of the innovations disclosed herein.

In addition, as used herein, the term “or” is an inclusive “or” operator, and is equivalent to the term “and/or,” unless the context clearly dictates otherwise. The term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

The term “comprising” as used herein will be understood to mean that the list following is non-exhaustive and may or may not include any other additional suitable items, for example one or more further feature(s), component(s) and/or element(s) as appropriate.

The terms “physical activity” and “activity”, as interchangeably used herein, will be understood to mean any action, activity, sport, or the like, requiring a physical output from a user or athlete. While various embodiments herein described may make reference to physical activities such as cycling, or downhill or cross-country skiing, the skilled artisan will appreciate that the scope of the disclosure is not limited to such examples. For example, the system and methods herein described may be applied, for instance, to speed skating, skateboarding, snowboarding, running, rowing, canoeing, kayaking, motorcycle racing, swimming, wheelchair endurance sports, luge, skeleton, ski jumping, or the like.

Furthermore, and in accordance with various embodiments, an athlete may, in addition to the physical body of the athlete, further refer to any clothing or equipment used in the performance of an activity. For instance, when referring to aerodynamic drag on a cyclist, it will be understood that reference to the athlete may include, among other elements, the cyclist's body, protective gear such as a helmet, and/or the cyclist's bicycle. Similarly, reference to a skier may, depending on context, also refer to, for instance, the skier's body, clothing, skis, boots, protective glasses, and/or helmet.

The term “environmental interaction parameter”, as used herein, will be understood to mean an environmental variable that may relate to an athletic output or performance of an athlete and/or associated equipment. For instance, for a cyclist, an environmental interaction parameter may refer to an aerodynamic drag resulting from the cyclist's body and/or cycle moving through air at a given speed and/or direction. Alternatively, or additionally, an environmental interaction parameter may comprise a parameter related to a coefficient of rolling resistance, a road slope, an acceleration, a drivetrain resistance, a change in elevation, a kinetic or potential energy, or other like parameter that may affect, for instance, a cycling velocity, rider power output, or rider output efficiency. Similarly, an environmental interaction parameter for a skier may comprise an air or wind resistance, a friction between the skier's skis and the ground, an air speed and/or direction, a change in air or ground speed, or the like.

In accordance with various embodiments, an environmental interaction parameter may be measured, calculated, inferred, or the like, from one or more sensors coupled with an athlete or equipment associated therewith. For instance, an in accordance with various embodiments, a sensor for acquiring an environmental interaction parameter may comprise one which measures, calculates, determines, or estimates a wind speed or aerodynamic drag force. A non-limiting example of such a sensor, also herein referred to interchangeably as a “nacelle”, may comprise a means of determining a coefficient of drag (Cd) multiplied by a frontal area (A) of an athlete to provide, for instance, an aerodynamic factor of an athlete and/or equipment comprising the product CdA. Such embodiments may comprise, for instance, an air speed and/or air density sensor, which may be directionally sensitive, a non-limiting example of which may be that disclosed by International Patent Application PCT/CA2020/050316, filed Mar. 10, 2020 and entitled “Airspeed sensor, system and airspeed monitoring process digitally implemented thereby or in relation thereto”, the entire contents of which are hereby incorporated herein by reference. In accordance with various embodiments, a nacelle may comprise a Motus Nacelle™.

An “athletic position”, as referred to herein, will be understood to mean a state, position, configuration, stance, motion, action, or the like, that may be assumed or performed by an athlete (or associated equipment) during the performance of a physical activity. For instance, a cycling athletic position may include a rider body configuration, non-limiting examples of which may include, but are not limited to, a rider body position in which the athlete is standing, has her hands on the top bar, hoods, or drops, is in an aerodynamic position, is sprinting, has her back up, horizontal, or down, is in a relaxed aggressive posture, or the like. Similarly, a skier athletic position may comprise, for instance, a tuck position, an action or manoeuvre to be performed during a turn, or the like. In accordance with various embodiments, an athletic position may be related to an athlete's body, optionally including one or more pieces of clothing or equipment, or may relate to one or more body parts. For instance, a body position may comprise a hip or torso angle, a leg or arm flexion, or the like. A body position may comprise a particular configuration, or a range of configurations (e.g. a particular body position may comprise a designated range of hip angles).

An “optimal”, “preferred”, or “desired” body position, as used interchangeably herein, will be understood to mean the body position from a set of possible body positions that provides the highest degree of a particular advantage. For instance, an optimal body position may be one that produces the highest cycling velocity for a given metabolic effort in consideration of aerodynamic forces and expected athlete output power.

An athletic position, in accordance with some embodiments, may be measured (directly or indirectly), inferred, or otherwise estimated using one or more sensors. For instance, an athletic position sensor may comprise one or more proximity sensors fixed to one or more parts of an athlete and/or equipment operable to infer, for instance, a torso angle with respect to the athlete's upper or lower body, equipment such as a bicycle, the ground, or the like. Similarly, a gyroscopic or other sensor operable to determine an athlete orientation may be used to infer a body position. Alternatively, or additionally, a directionally sensitive sensor, such as an air speed sensor oriented at a designated angle or operable to determine a direction of air flow may be employed to infer an athlete body position. In yet other embodiments, body position may be inferred, calculated, or directly measured using one or more images or a video of an athlete performing an activity.

An athletic position may be determined, in accordance with various embodiments, either in real time (or with minimal delay) during performance of the athletic activity, or after performance of the activity from sensor data stored and/or communicated to an external device for post-activity processing and/or reporting.

An “aerodynamic interaction metric”, as used herein, will be understood to refer to a measurable, quantifiable, estimable, or representative quantity that may relate to an athletic output or performance of an athlete and/or associated equipment with respect to an aerodynamic force. Further, and in accordance with various embodiments, an aerodynamic interaction metric may be related to or otherwise associated with one or more athlete positions. For instance, an aerodynamic interaction metric may refer to an effective cross-sectional area, drag coefficient, air or wind resistance, drag force, or combination thereof for a cyclist in a given body position on a bicycle. Additionally, or alternatively, an athletic interaction for a cyclist may refer to a coefficient of rolling resistance, a road slope, an acceleration, a drivetrain resistance, a change in elevation, a kinetic or potential energy, or other like parameter that may related to, for instance, a cycling velocity or rider power output for a particular athlete position. Similarly, an aerodynamic interaction metric for a skier may comprise a frontal area or coefficient of drag in a given skier position, or an amount of friction experienced during a turning manoeuvre.

While an aerodynamic interaction metric may be a predetermined value associated with one or more athlete positions, it may alternatively, or additionally, and in accordance with various embodiments, be measured, calculated, inferred, or the like, from one or more sensors coupled with an athlete or equipment associated therewith. For instance, proximity sensors located on, respectively, a knee and abdomen of an athlete, may be used to infer, for instance, a hip or torso angle, which may in turn be used to infer an effective cross-sectional area of the athlete's body, or to infer an athlete position (e.g. tuck position for a skier, sprint position for a cyclist, or the like). Furthermore, and in accordance with some embodiments, an aerodynamic interaction metric may be related to an environmental condition. For instance, a frontal area of a cyclist or skier may be combined with an environmental interaction parameter such as airspeed to estimate an aerodynamic drag force and associated work lost to drag for an athlete in a designated body position using an appropriate force model.

In accordance with various embodiments, one or more of an aerodynamic interaction metric, environmental interaction parameter, and/or athlete body position may be associated with an athletic output, such as an output power, energy, velocity, or efficiency. For example, a cyclist in a crouched position, while potentially more aerodynamic, may not output as much power over a designated duration of time as when that athlete is in an upright position, due to, for instance, physiological constraints. However, over a longer duration, the effects of aerodynamic drag may result in a net reduction of total power for the rider in an upright position compared to a situation in which the rider was crouched, such as if the cyclist were to tire due to the accumulated effects of increased drag. Accordingly, an athletic output, as herein described, may, in some embodiments, comprise aspects of a power-duration curve, an output efficiency, or the like, and may have a time dependence, and/or may be a function of, or may be represented in terms of, a metabolic cost, as further described below.

The systems and methods described herein provide, in accordance with different embodiments, different examples in which an optimal, or at least improved, athletic body position may be determined for an athlete performing an activity. Various embodiments further relate to directly or indirectly measuring or otherwise inferring an athlete body position during performance of an activity using sensors to determine an improved or optimised aerodynamic drag, either in real time during performance of a sport or activity, or post-activity to, for instance, indicate where an athlete could improve for a subsequent performance. In accordance with various embodiments, the systems and methods herein disclosed may further be employed to determine an optimal or improved body position in real time based on sensor data, and/or aerodynamic drag and/or power output models, and output a corresponding signal to the athlete to, for instance, perform an action or alter a body position to improve output and/or reduce loss due to aerodynamics. Accordingly, various embodiments may relate to improving athletic performance of an activity by optimising an athletic output (e.g. speed) in view of a metabolic cost associated with, for instance, a body position adopted to provide that output, and associated environmental interactions (e.g. aerodynamics).

As an exemplary embodiment, the following description relates to a summary overview of integrating data from a typical bicycle sensor suite, which may include one or more sensors to monitor a cyclist body position. However, it will be appreciated that such concepts may be applied for other activities, in accordance with other embodiments.

In a typical bicycle system, there is a balance of energy; namely, that the energy input into the system equals the energy out. The energy applied by a rider to push the pedals is ultimately utilised to overcome a combination of moving against a wind resistance, moving against a rolling resistance and drivetrain resistance, changing elevation (i.e. moving against or with a gravitational potential energy), and changing speed (i.e. changing kinetic energy). This balance of energies may be expressed as follows:

W_{Rider Power}=W_{Rolling Resistance}+W_{Elevation Change}+W_{Kinetic Energy}+W_{Aerodynamic Drag}

where the work provided by the rider may be equated to the work provided by the rolling resistance, elevation change, aerodynamic drag and kinetic energy.

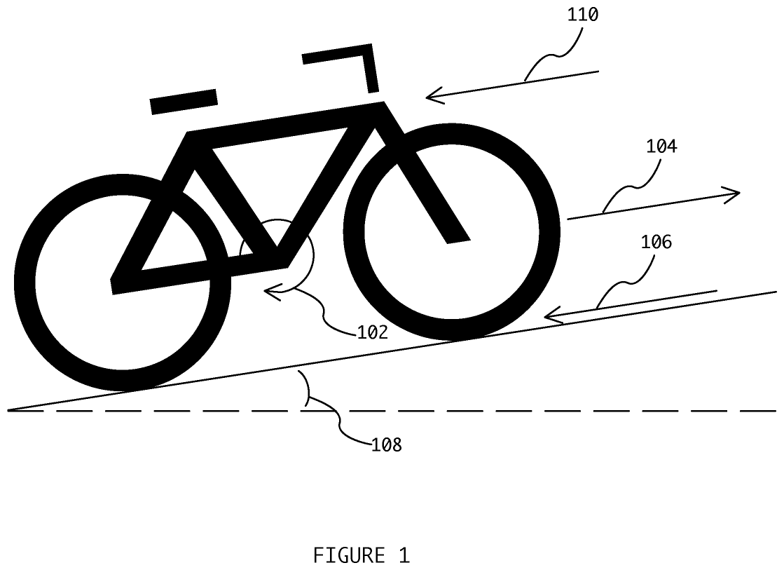

FIG. 1 provides a diagrammatical representation of the various forces/parameters at play in an illustrative cycling embodiment. Generally, the cyclist's input power 102 is schematically illustrated as a force conveyed through the drivetrain and commonly measured via one or more strain gauges mounted to the rear wheel hub, bottom bracket/spindle, chainrings and crank spiders, crank arms and/or pedals.

The cyclist's input power works to drive the bicycle and cyclist forward at a given speed (e.g. kinetic energy) 104, which can also be measured using common speed and/or cadence meters, or again via GPS or other positional or motion tracking systems.

Various external forces, however, will naturally impact the user's velocity (and acceleration), such as a rolling resistance 106, an elevation change or slope 108 (which of course acts in the cyclist's favour when rolling downhill), and aerodynamic drag 110. As noted below, while some of these aspects can be measured or calculated directly from sensor readings (e.g. slope or elevation change using onboard inclinometers, gyroscopes, accelerometers, etc.), others, such as aerodynamic drag, may be estimated or solved for using computational techniques, in accordance with different embodiments. Further sensors may nonetheless partake in these calculations, such as wind velocity sensors, for example, which can be used alone or in combination with ground speed readings depending on the sensors used and application at hand, to obtain relative air speed values useful in the computation of aerodynamic drag. Namely, the acquisition or computation of relative air speed values allows for the treatment of real-world conditions (as opposed to being limited to still air conditions), for example in gaining access to free stream considerations, and further accentuating scenario's like drafting, for example.

On this basis, the above-noted expression can be re-expressed as follows to accurately represent the cyclist's power and speed:

$${W}_{RP} = Crrmgv + smgv + mav + \frac{1}{2} CdA ρ {v}_{air}^{2} v$$

where:
W_{RP}=Rider output power (from power meter) (W)
   v=Speed (“ground speed”) (m/s)
   m=Total mass (rider+bike) (kg)
   g=Acceleration due to gravity (9.81 m/s²) (m/s²)
Crr=Coefficient of Rolling Resistance
   s=Slope of road (degrees)
   a=Acceleration of cyclist (m/s²)
   ρ=Air density (kg/m 3)
Vₐᵢᵣ=Air speed (ground speed+wind speed) (m/s)
Cda=Coefficient of drag×frontal area (m²)

As noted above, parameters such as ground speed, slope of road, air speed, rider output power, acceleration of cyclist and/or road speed can be directly or indirectly measured using appropriate equipment. Other parameters like gravitational acceleration, the mass of the rider and bike, and air density, can be readily input into the system.

It will be appreciated that this model can be altered to add or remove different terms given the application at hand, but also to further refine precision depending on the type of sensor used, its location and mounting mechanism on the user/vehicle, and/or other mechanical considerations at play. For example, a drivetrain efficiency estimate could be incorporated to account for drivetrain losses, for example, when a power measurement is made at the crank, some losses being expected in this context as the wheel is driven.

Using this expression and known, measured or estimated variables, one can then rearrange the terms to solve for acceleration and so also access computed solutions for velocity and position, as follows:

$$a = \frac{{W}_{RP}}{mv} - Crrg - sg - \frac{CdA ρ {v}_{air}^{2}}{2 m} \qquad ( 1 )$$

In order to solve for acceleration, each of the other variables may be initialized to realistic values based on gathered data and tests as well as actual measurements for parameters such as air density and rider mass. A differential equation solver may then be used to solve the equation, such as the MATLAB differential equation solver ODE45, though others may readily be employed, as may firmware be deployed for execution by an onboard microcontroller, for example.

In the results described below, the ODE45 solver was run on the input differential equation to produce solved values for acceleration, velocity, and position. Given a perfect data set, the state equation noted above could be solved directly to produce usable results, however, given the inherently noisy nature of the input measurements and the unpredictable variations in unknown variables such as aerodynamic drag variations, further consideration was required to achieve usable results.

For instance, and as noted above, to avoid sensor noise inadvertently misleading results, sensor noise was explicitly accounted for in computing more accurate results. For example, in one embodiment, to model signal noise on sensors such as the speed or power sensor, a zero mean normally distributed random value was added to the known values (i.e. the ODE45 solution for velocity, or initialized power value). In the example provided below, the variance of the signal noise was based on sensor accuracy provided from the manufacturer or observed variations in the case of power sensor data, for example, as previously observed using the Chung method. Below is a chart of model signal noise variance values used for signal noise modeling.

| | Signal Noise Variance | |
|---|---|---|
| Ground Speed Sensor | ∓0.25 | km/h |
| Air Speed Sensor | ∓1 | km/h |
| Slope Sensor | ∓0.5% | Grade |
| Rider Power Sensor | ∓5 | W |

To be able to track the CdA of a cyclist in real time with multiple sensors each with signal noise, a signal filtering solution may be pursued. The non-trivial selection and adaptation of a signal filter was required to address, amongst other items: the non-linearity of the state equation at hand (i.e. which includes a quadratic term; the existence of two unknown and immeasurable variables (CdA and Crr) in this particular example, though a greater number of unknown and immeasurable variables may be considered in this or other similar examples within the scope of the present disclosure; the input from multiple (possibly redundant) sensors each having sensor noise; and that the solution should ultimately be able to run in real time on a microcontroller for onboard use.

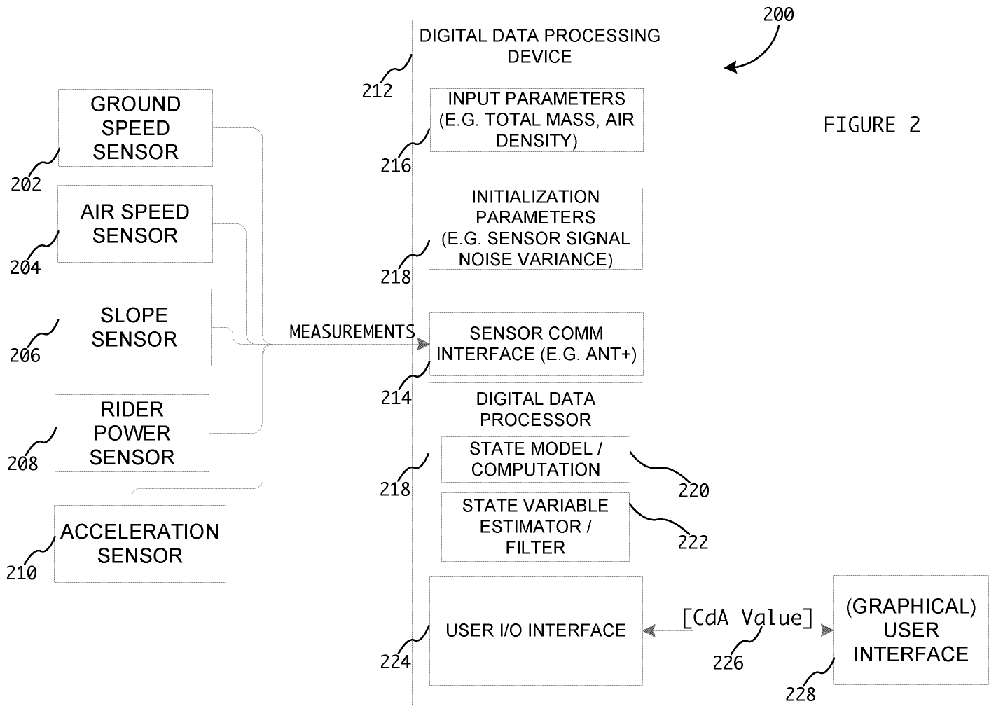

With reference to FIG. 2, a schematic diagram of an aerodynamic drag monitoring system, generally referred to using the numeral 200, will now be illustratively described. In the illustrated embodiment, the system comprises or is adapted to communicatively interface with one or more sensors, and generally a number of such sensors, that can be used as input variables to the cyclist's dynamic state model to estimate and/or compute various unknown variables such as an aerodynamic drag value or indication. In the illustrated embodiment, the system is configured to operate on readings acquired via one or more of a ground speed sensor 202, an air speed (or wind) sensor 204, a slope sensor 206 (e.g. inclinometer(s), gyroscope(s), accelerometer(s), etc.), a rider power sensor 208 and/or an acceleration sensor 210.

Data signals and/or values from each of these sensors are continuously or discretely (e.g. at a set data transfer rate) transferred to a digital data processing device 212 operable to process such signals. While device 212 is illustrated as distinct device, it will be appreciated that various sensors may be integrally formed or associated with the device 212 in a common form factor, as can be provided as a distinct device operatively communicating with one or more external (e.g. proprietary or third party) sensors. Accordingly, the device 212 will include one or more sensor communication interfaces 214 to interface with each of the internal and/or external sensors. Different communication protocols may naturally be invoked to implement this interface, as can different protocols be used for different sensors depending on the nature thereof, whether sensors are integrated or external, wired or wireless, etc. In the illustrated example, the device 212 may take the form of or include a microcontroller that is specifically programmed to interface with each sensor wirelessly using an ANT+ or like protocol, common for athletic/physiologic monitoring sensor communications. Other communication protocols may also or alternatively be considered, such as based on Zigbee, Bluetooth to name a few.

In one embodiment, the digital data processing device 212 may be configured to receive and/or store one or more input parameters 216 useful in subsequent computations, such as for example, but not limited to, the total mass of the rider and vehicle, air density, etc. While such input values may be useful, the systems and methods as described herein may also be configured to estimate any of these values which, in some embodiments, may provide for greater accuracy, for example, where a rider gradually consumes water from a mounted water bottle that will inherently reduce the overall weight, and like considerations. The system further initializes a number of initialization parameters 218, such as in the following example, but not limited to, initial sensor and estimated value error ranges (the former generally derived from manufacturer specifications), directional tracking boundaries useful in mitigating potential windup issues in tracked/estimated values, and identification of which parameters are to be influenced by actual sensor readings as opposed to pure estimated values.

Based on the initialized and input parameters, and the input of sensor data, a digital data processor 218 may be operated, based on a stored state model and computational process 220, to act on these values to filter and estimate various state variables 222 of interest. Processor outputs may then be directed to an input/output interface 224 to provide an output indication as to an unknown state variable of interest, such as a CdA value 226, index or indicia, for example. Process outputs may be stored and managed locally for further processing, output on a local (graphical) user interface, or again relayed via wired or wireless communications to an external or third-party interface 228.

In one embodiment, the digital data processor 218 is configured to implement an adapted Extended Kalman Filter process at 222, which was configured to address the particular conditions at hand, namely to integrate known measurements over time, each containing statistical noise and/or other inaccuracies, and to produce estimates for unknown variables such as CdA and/or Crr by estimating a joint probability distribution over the variables for each timeframe. The fundamental principles of Kalman and Extended Kalman Filters are well known in the art and therefore need not be replicated herein. Detailed descriptions can be found, for example, in the following references, the entire contents of which are hereby incorporated herein by reference (H. M. Paul Zarchan, Fundamentals of Kalman Filtering: A Practical Approach, American Institute of Aeronautics and Astronautics, 2000; H. S. Liyu Cao, “The Kalman Filter Based Recursive Algorithm: Windup and Its Avoidance,” in Proceedings of the American Control Conference, Arlington, V A, 2001). The interested reader may find further information related to the advantages of such methods and systems for cycling applications in International Patent Application WO 2019/200465 A1 entitled “Aerodynamic drag monitoring system and method”, the entire contents of which are hereby incorporated herein by reference.

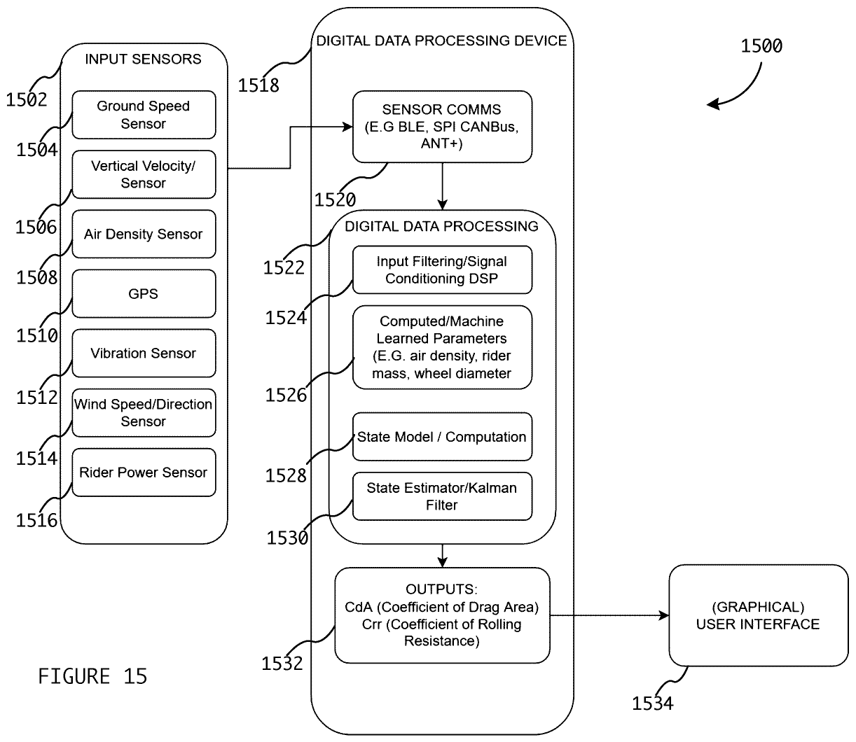

With reference to FIG. 15, a schematic diagram of an aerodynamic drag monitoring system, generally referred to using the numeral 1500, will now be illustratively described. In the illustrated embodiment, the system 1500 comprises, or is adapted to communicatively interface with, one or more input sensors 1502, non-limiting examples of which may include a ground speed sensor 1504, a vertical velocity sensor 1506, an air density sensor 1508, a GPS 1510, a vibration sensor 1512, a wind speed and/or direction sensor 1514, a rider power sensor 1516, or the like. Data from input sensors 1502 may be communicated to a digital data processing device 1518 via one or more sensor communication interfaces 1520 (e.g. BLE, SPI CANBus, ANT+, or like interfaces) associated therewith for processing by a digital data processor 1522. The processor 1522 may in turn comprise modules or processes for, for instance, input data filtering, signal conditioning, or digital signal processing 1524, and may additionally or alternatively comprise computed and/or machine-learned parameters 1526 related to, for instance, air density, rider mass, wheel diameter, or the like. The processor 1522 may, in accordance with various embodiments, comprise various state models 1528, state estimators 1530, and/or filters 1530 for processing input data. The processor 1522 may further provide output 1532 related to, for instance, CdA, Crr, or the like. A signal related to, for instance, aerodynamic drag or rolling resistance may then be output from the processing device 1518 for display to an athlete or coach via, for instance, a graphical user interface 1534.

While riding in varying conditions, the proportion of power (i.e. energy per unit time) utilised in overcoming each of various components varies. For example, climbing a hill may primarily change the gravitational potential energy of a bike/rider, while riding on the flats may primarily comprise overcoming wind resistance.

Further, there is a metabolic (physiological) relationship between rider power output and position. The biomechanics of pedalling a bicycle are such that a rider may be able to physiologically generate and sustain more power as they sit more upright and open, and less power as they sit with their torso leaning more forward. This is often referred to as metabolic cost or metabolic effort.

While riding a bicycle in competitive situations, typical goals of the rider are to either travel as quickly as possible for a desired metabolic effort, or to expend the minimum metabolic effort to sustain a desired speed. If the above relationships are defined for a given rider, at every instance in a changing environmental landscape, there is a preferred position that may be calculated that may maximise the speed at which a rider will travel for a given physiological effort. In accordance with various embodiments, the systems and methods herein described relate to determining this body position using, for instance, the abovementioned energy balance relationships, and in some embodiments, relaying information related to this body position to a rider in real time.

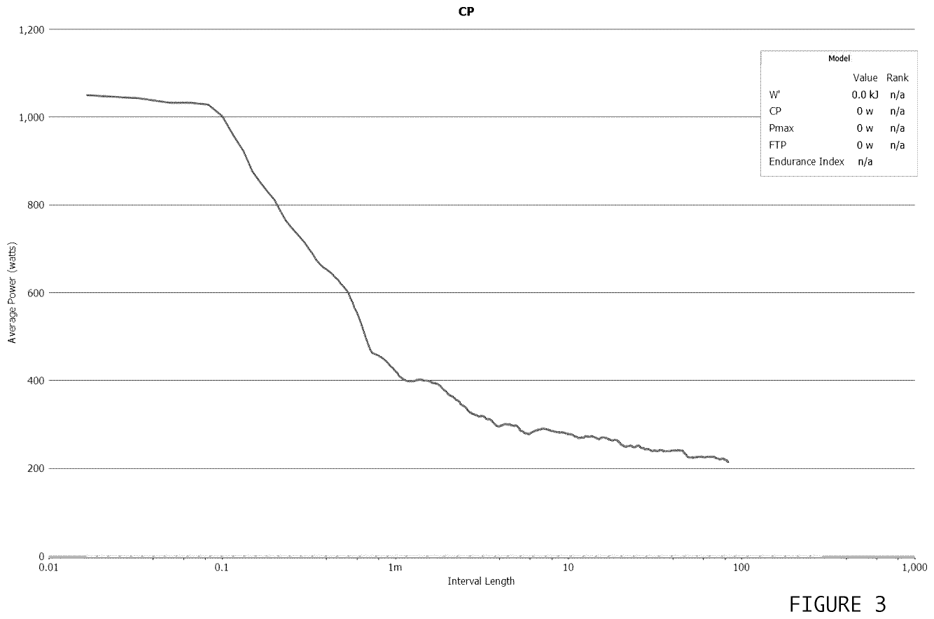

A common analysis metric used in a variety of sports, including, but not limited to, endurance sports such as cycling, cross-country skiing, and speed skating, is the power-duration curve, an example of which is shown in FIG. 3. Such curves typically represent the amount of power a rider is capable of sustaining over various durations of time. Typically computed by tracking rider data over several rides, the average power sustained over a sample window of a fixed time duration may be computed for a number of time windows available in a data set. In accordance with various embodiments, the maximum average power output found for that time duration may be treated as a “benchmark”, and this process may be repeated for sample windows of varying durations to build a more complete curve. Further, and accordance with some embodiments, the duration that an athlete may be capable of sustaining a given output power level may be viewed as a measure of the metabolic effort required to sustain that power level.

As an exemplary embodiment of the methods and systems herein described, the relationship between output power, or a power duration curve, athlete body position, metabolic effort, and aerodynamic forces will now be described in the context of a cyclist riding a bicycle. For the purposes of illustration, only, discussion will relate to the common cycling positions of standing, hands on top (very relaxed), hands on hoods (upright or relaxed), hands on drops (aggressive), and the “aerodynamic position”, although it will be appreciated that other cycling body positions, as well as body positions in other sports or activities, are also contemplated by the systems and methods herein disclosed.

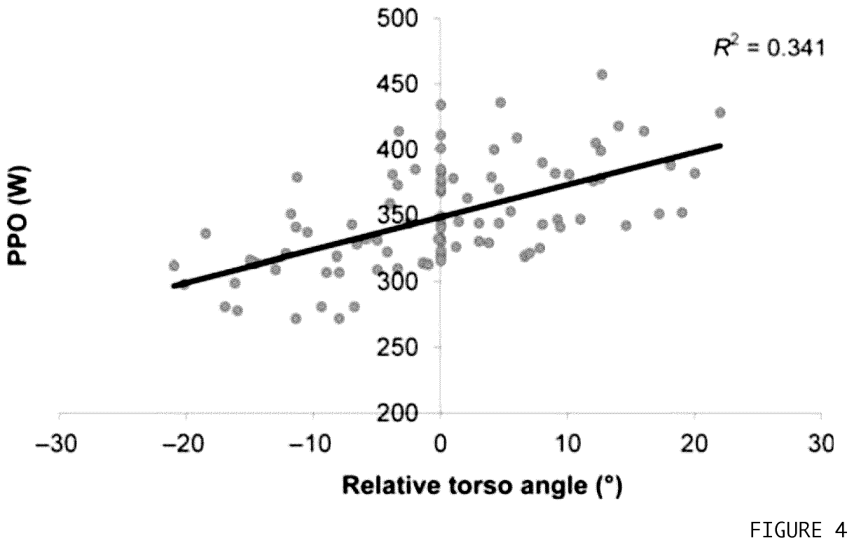

While variations exist between athletes based on factors such as body shape, flexibility, comfort, torso angle, hip angle, shoulder angle, and the like, there is generally a link between body position and the metabolic effort required to sustain a given power output. For example, FIG. 4, adapted from Fintelman, et al. (“The effect of time trial cycling position on physiological and aerodynamic variables,” Journal of Sports Sciences 33(16):1-8, 2015) shows the proportional power output of a group of 39 cyclists as a function of torso angle. In this example, a typical difference of almost 100 W in maximal 3-minute power output from a fully upright to fully bent over position was found.

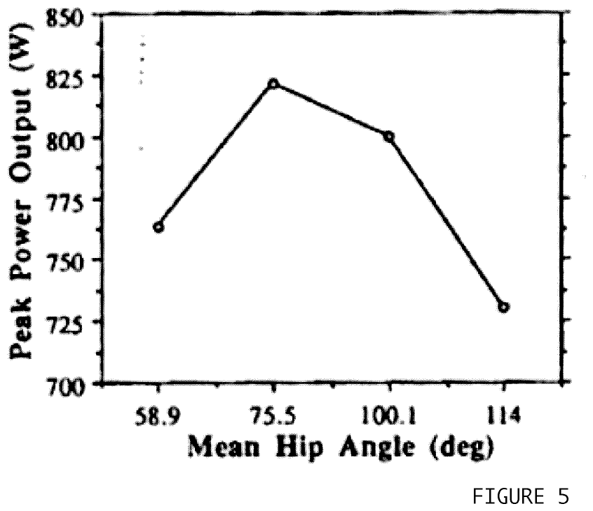

On the other hand, FIG. 5, adapted from Too (“The Effect of Hip Position/Configuration on Anaerobic Power and Capacity in Cylcing,” International Journal of Sport Biomechanics 7:359-370, 1991) shows a non-first order relationship between power output for a cyclist as function of mean hip angle. As such, and in accordance with various embodiments, various relationships or functions may be established, recognised, modeled, or assumed between a power output (or peak power output) of an athlete and a body position thereof. Further, various embodiments relate to body position-related power output functions that have been established or characterised using various sources. For instance, while some embodiments relate to models generated from aggregate statistics of, for instance, controlled studies of a plurality of athletes, other embodiments relate to parameter relationships determined from, for instance, machine learning methods, and/or tracked data from an individual athlete who performed an activity one or more times.

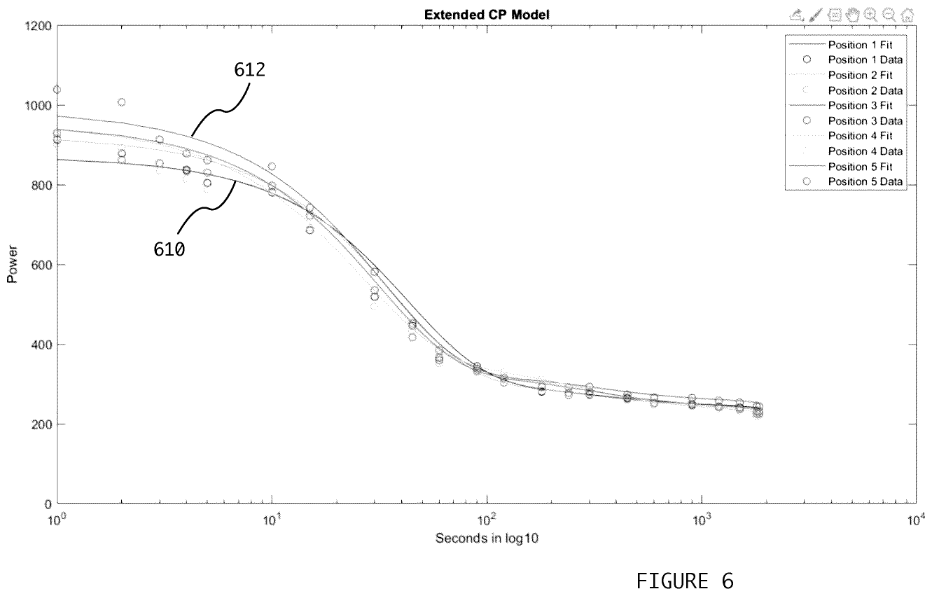

Extending the relationship between athlete output power and body positions, illustrated for exemplary purposes in FIGS. 4 and 5, there also exists a relationship between the power duration curve of an athlete (or plurality of athletes, via a mean or other statistical relationship) and body position during a sport or activity. This concept is illustrated in FIG. 6, where exemplary power duration curves for five different athlete body positions are shown. These represent the duration that a rider is able to sustain a given power level for a given body position. Curve 610 represents a first body position that is the most bent over of the five examples shown. While it has the lowest output power for durations up to approximately 10 seconds, it also has the highest power in a time window of approximately 30 seconds to 90 seconds. Curve 612, on the other hand, represents the most upright position of the five examples, having the highest output for durations up to approximately 20 seconds, but a lower power than other positions for durations up to approximately 300 seconds.

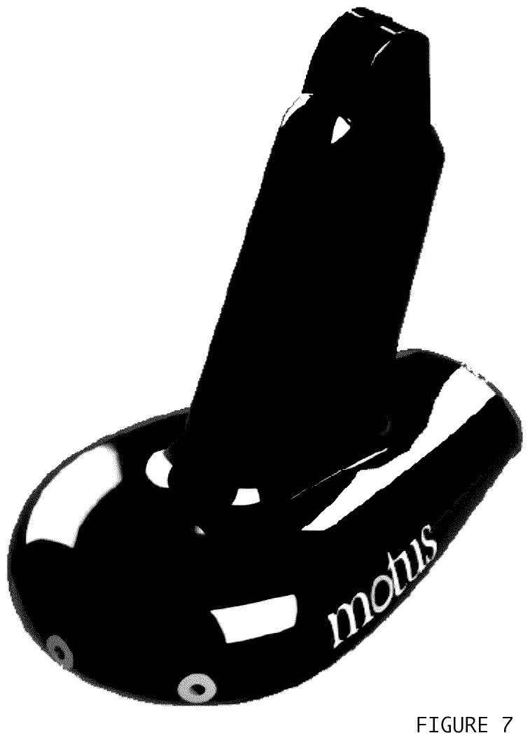

Various embodiments of the systems and methods herein disclosed relate to the determination of various aerodynamic factors that may affect athlete performance. One example of such an aerodynamic variable is the coefficient of drag (Cd) multiplied by a frontal area (A) of an athlete, the product of which is herein represented by the variable CdA. This may, in accordance with various embodiments, be used to determine how much drag force is experienced by an athlete, such as a cyclist or skier, or on a body in flow. This may be measured or inferred using, for instance, a device such as a “nacelle”, an example of which is shown in FIG. 7. The interested reader may find further information and sensor examples for determining, among other aerodynamic parameters, CdA in International Patent Applications WO 2019/200465 A1 and PCT/CA2020/050316.

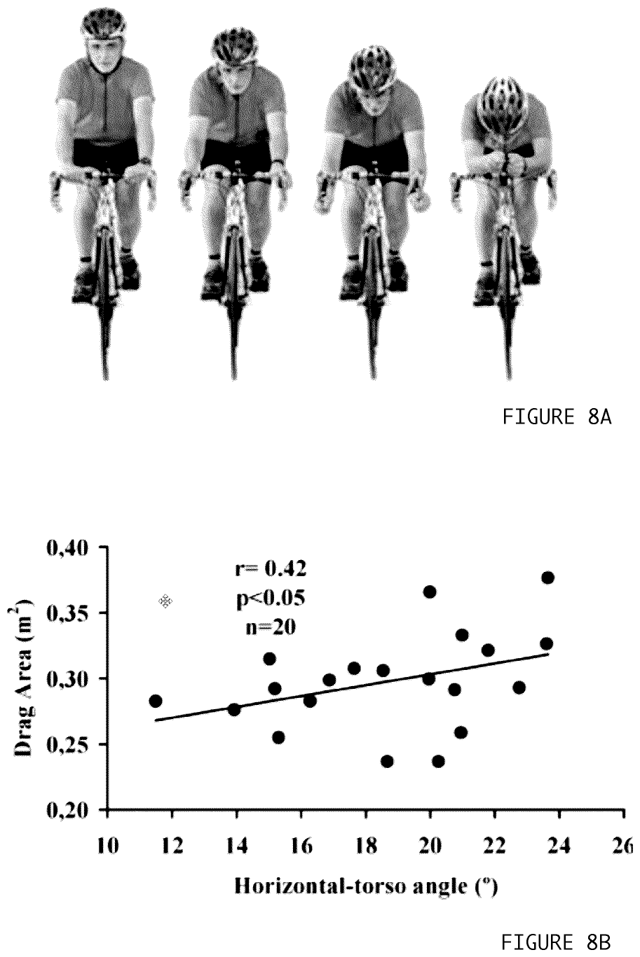

The frontal area may in some embodiments comprise the cross-sectional area taken up by, for instance, a cyclist, from a particular angle, such as in front of the athlete in line with the direction of motion. This may be related to the body position of the athlete, examples of which are illustrated in FIG. 8A, where four exemplary body positions of a cyclist are shown. From left to right in FIG. 8A, the images of the different body positions show a decreasing frontal area.

While various embodiments relate to the determination of frontal area using images of a rider such as those shown in FIG. 8A, various other means exist for determining A or CdA for a rider in a given position. For instance, the body area of an athlete may be calculated or inferred from various measurable body metrics and/or joint angles. FIG. 8B, adapted from Garcia-Lopez, et al. (“Reference values and improvement of aerodynamic drag in professional cyclists”, Journal of Sports Sciences 26(3):277-286, 2008), shows one such example, where the drag area of an athlete's body is shown as a function of torso angle from horizontal.

Similarly, there exists a dynamic relationship between an athlete's body position and coefficient of drag (Cd) that may contribute to the relationship between CdA and athlete body position. Exemplary values of Cd, A, and CdA are shown in the following Table, which, in accordance with some embodiments, may serve as assignable values in computational models and/or calculations when determining, for instance, an optimal or preferred body position with respect to aerodynamics for an athlete in a given activity and/or environmental circumstance (e.g. when travelling at a particular speed and angle relative to air).

| Position | A (m²) | C_{d} (—) | C_{d}A (m²) |
|---|---|---|---|
| Sprint regular | 0.460 | 0.670 | 0.308 |
| Sprint low | 0.374 | 0.626 | 0.234 |
| Back up | 0.423 | 0.655 | 0.277 |
| Back horizontal | 0.370 | 0.638 | 0.236 |
| Back down | 0.339 | 0.655 | 0.222 |

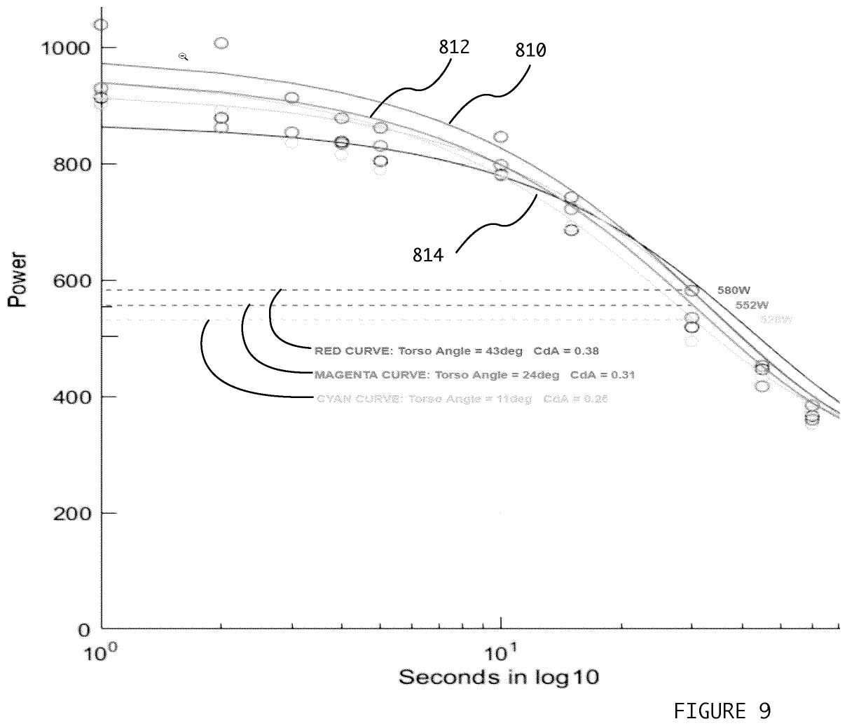

Given the relationship between aerodynamic drag and body position, it is therefore not necessarily the body position that would result in the highest output power that may be desirable or optimal for a given time duration when one considers potential energy or power losses to aerodynamics. For example, FIG. 9 shows the power duration curves from FIG. 6, zoomed to approximately the first 30 second time window. Dashed lines are guides to the output power for a 30 second duration for the “RED CURVE”, “MAGENTA CURVE”, and “CYAN CURVE”, shown as elements 810, 812, and 814, respectively. While the red curve 810 shows the body position with the highest output power at 580 W, it also has the highest CdA value of 0.38, which may result in the most aerodynamic drag. The body position represented by the cyan curve 814, on the other hand, has the lowest output power of 526 W, but may result in the least amount of drag with a CdA of 0.26. If a rider were to be in an upright position (e.g. the body position corresponding to curve 810), it may be desirable in certain situations to have a lower “desired effort” output for, for instance, a 30 s duration, and assume a more bent over position, such as that represented by curves 812 or 814. Conversely, a tail wind may interact productively with an athletic body position that relates to a high CdA value.

For athletic activities such as cycling, an athlete may have one of several goals. For instance, and in accordance with various embodiments, it may be desirable to travel as quickly as possible for a given physiological effort, or it may be preferable to expend the minimum physiological effort to sustain a given speed. As such, various aspects of the systems and methods herein disclosed relate to determining body positions that may be preferred when performing an activity to achieve various goals.

Returning to the concept of the energy and power output when performing an activity (e.g. cycling), the balance of energies equation that may be employed in accordance with various embodiments is reproduced here for clarity:

$${W}_{RP} = Crrmgv + smgv + mav + \frac{1}{2} CdA ρ {v}_{air}^{2} v$$

In accordance with various embodiments, a sensor suite, such as one that may be on board a nacelle (e.g. a Motus Nacelle™), may be operable to directly measure, compute, or infer many or all of the variables relevant to a particular situation. However, and in reference to embodiments related to cycling, riding in varying conditions may vary the proportion of power (energy per unit time) utilised in overcoming each of the energy-related components. For example, while climbing a hill, a cyclist power may by primarily affected by a change in gravitational potential energy of the rider and bicycle. Conversely, while riding on relatively flat terrain (i.e. “the flats”), rider power may relate primarily to overcoming wind resistance. As there is a trade-off between physiological power and wind drag, the optimal or desirable position at any given moment may be one that is determined from one, the other, or various combinations of these two or more contributions.

Continuing with the example of three distinct positions discussed with respect to FIG. 9, a rider may find that they are currently in a position represented by the “RED CURVE” 810 with a torso angle of 43 degrees, with a corresponding CdA of 0.38 and a power of 580 W. These parameters will contribute to the current velocity that an athlete and equipment (e.g. a cyclist and bicycle) are travelling.

In accordance with various embodiments, a system (or method employed thereby) may evaluate a velocity at which an athlete may be able to travel for a set of measured or inferred variables (e.g. wind conditions, terrain grade, rolling resistance, etc.) by solving, for instance, the balance of energies equation for a designated power and CdA, for a variety of possible body positions. From these solutions, it is possible to determine, for instance, the fastest velocity that the athlete can achieve, which in turn may, in some embodiments, define an optimal or preferred power duration curve and/or associated body position that may maximise speed for a particular metabolic effort.

Conversely, and in accordance with various other embodiments, an optimal or to preferred body position may be determined by finding and/or calculating the points on a set of power duration curves corresponding to different body positions that would result in a given speed and/or velocity that provides the longest duration (i.e. lowest metabolic cost) to maintain that speed. In accordance with some embodiments, these two methods may be equivalent, and may result in the same optimal or preferred athlete body position. However, various distinct power duration curves corresponding to various body positions may be expanded in, for instance, resolution, depending on the application and/or needs of a user.

Various embodiments relate to the use of various metrics that may be measured or inferred to define a preferred body position for an athlete in consideration of, for instance, output power and related aerodynamics. For example, various embodiments relate to different methods and systems for determining a speed or range of speeds that is accessible to an athlete in various body positions while maintaining a designated metabolic effort and/or performing the activity for a designated amount of time. To this end, various relationships between one or more power duration curves, body positions, CdA, and the like, may be established for one or more athletes. Such relationships may be, for instance, modeled or learned from tracking a specific athlete over one or more performances of an activity, and/or may be representative of group of athletes or aggregated statistics corresponding thereto.

Moreover, as described above, such concepts may by beneficially applied across different activities or athletic disciplines, in accordance with various embodiments. For example, sports such cross-country skiing, speed skating, rowing, running, canoeing, kayaking, wheelchair endurance sports, or the like may utilise quantifiable relationships between, for instance, body position and CdA or Crr, and body position and metabolic cost of producing power, to improve athletic performance and/or output. Similarly, sports such as downhill skiing, snowboarding, luge, skeleton, motorcycle racing, ski jumping, or the like may benefit from various embodiments herein disclosed to learn or improve understanding of relationships between CdA and body position, ultimately enabling improved coaching and/or performance of activities via observed or real-time measurements of aerodynamic parameters (e.g. CdA). In accordance with yet other embodiments, the systems and methods herein described may be further applied to activities performed using powered equipment. For example, and without limitation, one embodiment relates to electric bikes, wherein a method as herein described may be employed to provide an overall efficiency of both human power and electric battery power delivered to optimise an output, such as a maximised speed per watt.

As an illustrative example of how one may apply the concepts herein described to other activities, one may consider aspects of cross-country skiing that are similar or analogous to those described above with respect to cycling. For instance, in order to determine a propulsive power (e.g. rider power W_{RP}) from a cross-country skier, one may employ an instrumented ski binding and a sensor on the skier's poles configured to measure propulsive forces therefrom. Such data may be considered in combination with data from a GPS or accelerometer measuring forward velocity to determine propulsive power W_{skier power} as the measured force multiplied by velocity. A cross-country skier may further experience energy losses analogous to the rolling resistance of a bicycle (i.e. W_{rolling resistance}) as sliding friction between the snow and skis. The work associated with elevation changes may further be accounted for via a sensor (e.g. a Nacelle sensor) mounted to the skier (e.g. as a head-mounted sensor) and operable to measure elevation changes, the data from which may be combined with skier and equipment weight (e.g. as measured prior to performance of the activity). Similarly, as the velocity and mass of the skier and equipment may be known, the kinetic energy component of energy balance relationships above may also be known. Using a nacelle, as described above, one may further capture environmental parameters (e.g. wind speed and direction, air density) to calculate energy or work associated with aerodynamics (i.e. W_{aerodynamic drag}).

It will be appreciated that such aspects may similarly be readily applied to other activities. For example, a speed skater may be equipped with instrumented skates operable to measure propulsive forces, a nacelle sensor mounted on a helmet or the skater's body to sense aerodynamic parameters, and the like to determine similar work- or energy-related parameters.

Determination of such parameters may enable the determination of CdA and Crr, as described above with respect to cycling. Similarly, body position may be inferred using inertial sensors (e.g. IMUs, accelerometers, gyroscopes, magnetometers, or the like) in combination with a body position model (e.g. a constrained skeletal model) and orientation determination process known in the art. The relationships between body positions and various energetic considerations may then be applied to improve an athletic output or performance.

For instance, with respect to the exemplary embodiment of cross-country skiing, which generally relates to a cyclical body ‘stroke’ movement, one may consider, for instance, two aspects of body position: the overall ‘average’ body position during a full cycle of motion that results in an average aerodynamic drag, and the cyclical movement itself, which may characterise skier ‘technique’. In accordance with some embodiments, the combination of these aspects (i.e. the average body position and a cyclical average in technique during all phases of a ski stroke) may be used to classify an athlete body position, similar to how body positions may be classified for cycling, as described above (e.g. classified for cycling as ‘standing’, ‘hands on top’, ‘hands on hoods’, ‘hands on drops’, ‘aerodynamic position’, and the like). For clarity, body positions with respect to skiing are herein referred to as ‘body techniques’, although it will be appreciated that these terms may be used interchangeably herein.

It will be appreciated that different embodiments may relate to the inclusion of different energy parameters when considering improved or optimal body positions, depending on the application at hand. For example, and in accordance with one exemplary embodiment related to skiing, each body technique may be correlated or associated with aerodynamic drag, as well as an overall effective drag from sliding resistance. For instance, skiing may comprise non-propulsive elements of movement that result in an ‘effective sliding resistance’, such as the component of the ski stroke that pushes a ski sideways with respect to the direction of travel. Unlike cycling, in which body position has little impact on a rolling resistance, activities such as skiing may have a stronger or more significant relationship between body and energetic losses due to, for instance, sliding. Accordingly, for some applications, a body economy may essentially manifest as an effective sliding or like resistance. Improved body technique may therefore result in less loss of energy due to such effects, thereby improving an athletic output or performance.

In accordance with various embodiments, and as described above with respect to cycling, a metabolic cost associated with cross-country skiing or another activity may be quantified as a function of or in terms of an athletic output (e.g. power duration curve) for each body technique. Accordingly, various embodiments related to non-cycling applications may similarly proceed as, or comprise elements similar to, those described with respect to cycling for optimising or improving a body technique or body position in consideration of an athletic efficiency in terms of, for instance, an athletic output (e.g. speed) as a function of metabolic cost.

The following terms are hereby defined to help illustrate various embodiments:
(N)=current sample
(T_{N})=sample period
speed_{cur}(N)=the current speed that an athlete is travelling at the current sample (N)
speedₘₐₓ(N)=the maximum speed that an athlete could achieve in current conditions for a designated metabolic effort in the optimal or preferred position. The maximum speed may, in accordance with some embodiments, be determined by solving a balance of energies equation for one or more modeled or learned rider positions, given a desired or current duration or effort.
speedₘᵢₙ(N)=the minimum speed that an athlete would achieve in current conditions with a designated or current effort in a least or lesser optimal position. The minimum speed may, in accordance with some embodiments, be determined by solving a balance of energy equation for one or more modeled or leaned rider positions, given a desired or current duration or effort.
speedₗₒₛₜ(N)=speedₘₐₓ(N)−speed_{cur}(N)=the difference, at the current sample time, between the current athlete speed and the speed in an optimal or preferred position.
timeₗₒₛₜ(N)=(distanceₘₐₓ(N)−distance_{cur}(N))/speed_{cur}(N)=(speedₘₐₓ(N)*T_{N}−speed_{cur}(N)*T_{N})/speed_{cur}(N)=time lost (i.e. how much time is being lost by having a current athlete body position compared to having an optimal or preferred body position for a given time or sample interval, based on the relationship of time=distance/speed.
timeₚₒₜ(N)=(distanceₘₐₓ(N)−distanceₘᵢₙ(N))/speedₘᵢₙ(N)=(speedₘₐₓ(N)*T_{N}−speedₘᵢₙ(N)*T_{N})/speedₘᵢₙ(N)=potential time lost should an athlete be in a least optimal or preferred body position relative to an optimal or preferred body position for a given time or sample interval.

$$Aerosmart Score = {\sum }_{N = 0}^{current} {time}_{lost} / {\sum }_{N = 0}^{current} {time}_{pot} = a cumulative average of how much time an athlete lost over a designated time , interval , or sample compared a potential time lost in an optimal or preferred body position .$$

In accordance with various embodiments, an Aerosmart Score may assume various metrics to determine, for instance, how efficiently an athlete performed during an activity or portion thereof. For instance, an Aerosmart score may represent an accumulated lost time relative to what a particular athlete may have achieved over that same span of time in an optimal or preferred body position. As such, some embodiments relate to the reporting of a metric that is normalised to a particular athlete, rather than to a raw performance value. For example, an experienced or particularly skilled athlete may outperform a lesser athlete, even when performing the activity with a relatively low efficiency (e.g. a high-level cyclist having ridden in a body position that was far from optimal in consideration of aerodynamics may still ride a designated distance at a relatively high speed). In such a situation, the skilled athlete may have travelled a given distance in less time than a lesser athlete, even if the latter was more efficient in consideration of aerodynamics. However, various embodiments relate to an Aerosmart score that represents a rider efficiency in consideration of aerodynamics, which may report or otherwise reward the athlete who performed the most efficiently, regardless of their overall speed. As such, an Aerosmart score, in accordance with various embodiments, may comprise a feedback scoring system that athletes may use for relative comparison, even if there exists a discrepancy in talent or skill when using conventional metrics. As such, various embodiments relate to systems and methods of determining, quantifying, or inferring an athlete efficiency that is agnostic to the specific conditions of an athletic performance.

Various reported metrics, in accordance with various embodiments, may have various weights or relative importance based on designated goals or desired levels of efficiency. For instance, a reported metric may rank the importance of an optimal or preferred athlete position (e.g. a cyclist adopting the stance on a bicycle that provides the most optimal balance of power output and aerodynamic drag of a set of potential body positions) based on the amount of potential time and/or speed gained or lost with a particular choice of body position relative to that determined to be one that is more optimal or preferred.

The systems and methods described herein may further relate to the feedback of athlete efficiencies with respect to athlete body positions, power output, and/or aerodynamics that may comprise real-time feedback during the performance of a sport or activity, or the post-performance characterisation of the athlete. For instance, various activities may relate to fast-paced actions performed by the athlete during which sensors acquire data for post-processing. For example, a slalom skier may carry a nacelle that acquires/stores data related to air speeds, friction, and various kinetic energies, while a camera may record video of the skier navigating a course that is synchronised with data acquisition by other sensors. In such an embodiment, processing of the sensor data in light of skier body position as characterised from a video may be used to assess the skiers choice of body position (e.g. tuck, leg extension during a turn, etc.) during the run in order to report, between runs, areas and/or times during the performance where a more optimal body position may have been adopted to, for instance, save time.

Conversely, sensor data may be analysed in real time or with minimal delay by one or more processors on board, for instance, a nacelle, to give an athlete feedback on how she may improve a body position to be more efficient. For instance, a nacelle worn by a cyclist (e.g. a Motus Nacelle™) may be operable to determine an airspeed and direction relative the cyclist, as well as receive as input related to a terrain grade and elevation, at any time during a ride to determine what an optimal body position would be for that particular cyclist (or for, for instance, the average cyclist) given the environmental conditions. A device may then be operable to output information related to that optimal or preferred position in real time as an indicator to the athlete of what body position would, for instance, be most efficient.

While some embodiments relate to the communication to an athlete of what body position may be the most efficient for a particular goal (e.g. maintaining a velocity at a minimum metabolic effort, or achieving the maximum velocity for a given metabolic effort for a particular duration, etc.), yet other embodiments relate to systems and methods for characterising a current athlete performance with one that is more optimal or preferred given various measured or inferred parameters, either in real time or as a means of providing feedback between athletic activities. For instance, sensors on an athlete's body may be employed to determine a cyclist's current body position. Algorithms on board a nacelle or system communicating therewith may be operable to assess or compute based on stored rider data how efficient the current body position is in consideration of measured or inferred aerodynamic parameters and power outputs accessible to the athlete for a given duration of time. The system may then determine, for instance, a degree of inefficiency based on the rider's current body position relative to a preferred one, or may indicate a degree to which the athlete should deviate from their current state. Further, a system may indicate various computed parameters related to an efficiency, providing real-time feedback on, for instance, an amount of time and/or speed to be gained/lost by assuming a more optimal body position.

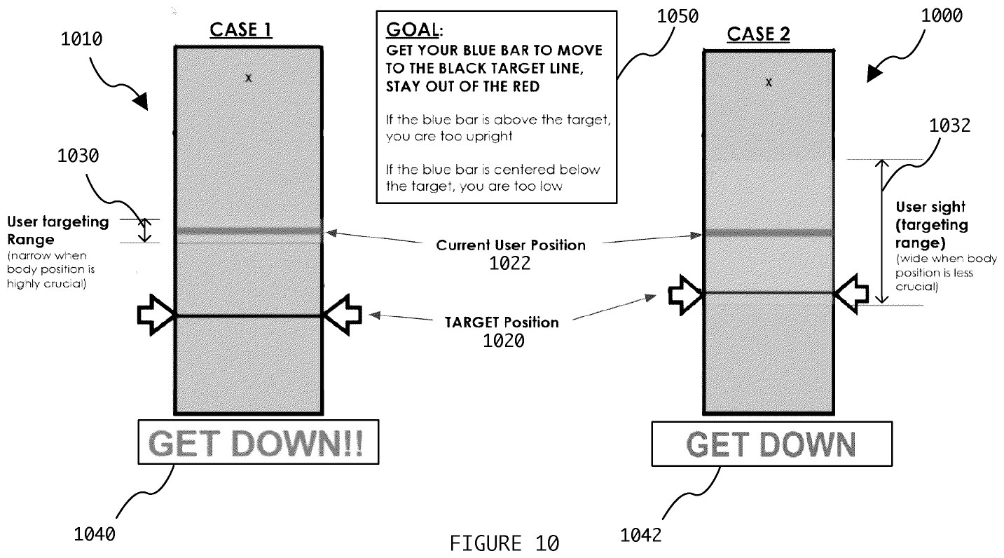

For instance, and in accordance with various embodiments, FIG. 10 shows an exemplary user interface 1010 operable to display to an athlete or coach feedback related to an athletic body position as determined from environmental aerodynamic variables and various related athletic parameters, as described above. In this example, a digital processing system may receive as input data related to, for instance, cycling conditions, such as an air speed and direction, road grade, data related to a cyclist frontal area, CdA, or the like, and compute, based on power duration models, CdA models, and/or stored athlete data, an optimal or preferred body position for the athlete. In accordance with some embodiments, an indicator for the preferred or target position 1020 may be representative of, for example, a body height or angle. Similarly, in embodiments where a current athlete body position is acquired, a similar indicator of the current user position 1022 may be displayed on the interface 1010. In some embodiments, such as if a body position (e.g. torso angle) will not drastically affect a rider output or efficiency, an interface 1010 may display a narrow targeting range 1030, about either the target position 1020 or current position 1022. Conversely, if there is significant potential for improvement, such may be indicated on the display 1010, such as by a wider sight targeting range 1032. Additionally, or alternatively, a display 1010 may indicate a recommendation or direction to the user, such indicators 1040 and 1042 instructing the user to alter their body position. Depending on the application, user instructions 1050 may further be displayed for, for instance, ease of use.

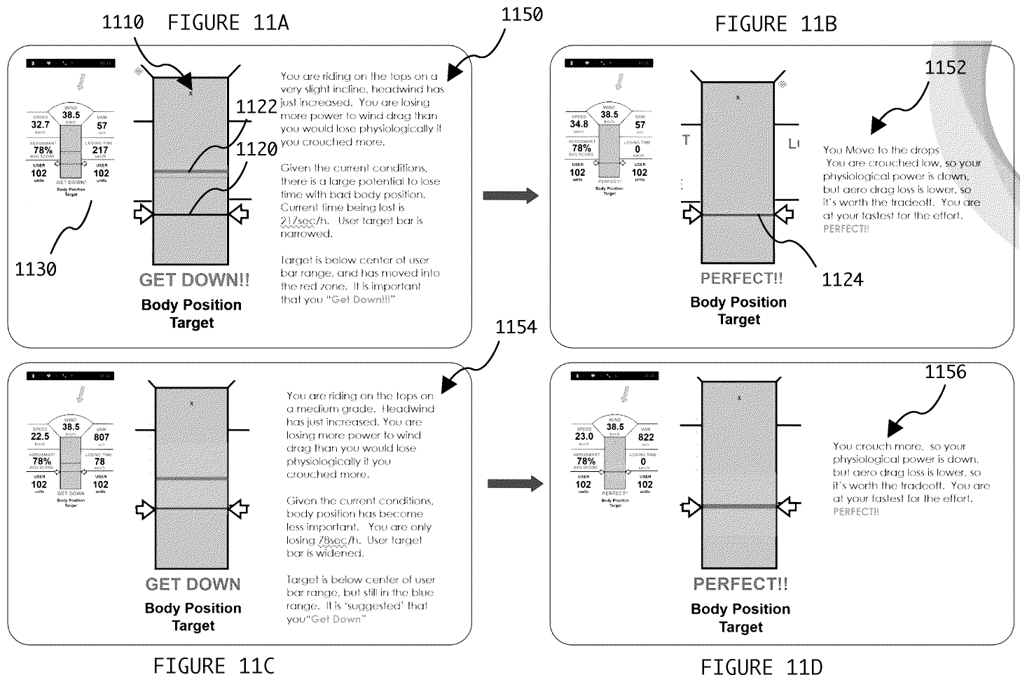

Similarly, FIGS. 11A to 11D show exemplary user interfaces for displaying feedback to an athlete or trainer. In these examples, each of FIGS. 11A to 11D show an indicator bar 1110 configured to display information related to an athlete body position as computed from a system or method as herein described. An indicator 1110 may display a target body position via, for instance, a line 1120. While these examples show a one-dimensional body position indicator, as may be useful in, for instance, representing a hip or torso angle, the skilled artisan will appreciate that other indicators may be employed within the scope of the systems and methods herein described. In this case, and in accordance with embodiments that measure or infer a current athlete position, a current status indicator 1122 may be shown on the indicator 1110 to provide information or highlight differences related to the current body position and a target optimal or preferred body position.

Again, the skilled artisan will appreciate that various metrics, information, instructions, and the like, may optionally be displayed to provide feedback, either in real time during or after performance of an activity. For instance, each of FIGS. 11A to 11D show an alternative display 1130 that may comprise an indicator bar such as indicator 1110, but also various other measured, calculated, or otherwise inferred parameters. For instance, a display 1130 may show information and/or parameters related to athletic performance modeling. Non-limiting examples shown in FIGS. 11A to 11D include data related to body position modeling of a cyclist, such as an air speed and direction, a cycling speed, a ground speed, an Aerosmart score, an amount of time that is being lost due to a sub-optimal body position, a user unit-based score, a visual cue as to the difference and relative importance of body position relative to a preferred athlete state, and an instruction to the athlete on how to improve body position in consideration of output power and aerodynamics. Depending on the application and/or activity, various levels of detail may be presented to the user. For instance, a display 1110 may provide a description 1150, 1152, 1154, and 1156 of parameters that are measured, inferred, and/or calculated. For instance, a description may include information input to a model or algorithm related to the user's current determined body position, a surface inclination, and a measured change in airspeed, and a general recommendation as to an improved body position. Further description, in accordance with various embodiments, may relate to a potential for improvement and severity of a particular body position relative to a preferred position, the relative importance of aerodynamics, and/or an urgency of a recommendation (e.g. “Get Down!”, “Get Down”, “Stand Up”, etc.).

As described above, feedback may be provided to the athlete in real time based on, for instance, a measured or inferred aerodynamic parameter. For instance, the indicator in FIG. 11A shows that there is a relatively large gain to be made by assuming a more crouched body position. As the athlete crouches down, the indicator may show an overlap 1124 as the current and target positions align. Conversely, if a potential gain is minimal, as shown in FIG. 11C, there may be little relative change reflected in an indicator as the athlete improved their body position, as shown in FIG. 11D.

Displays such as those in FIGS. 10 and 11A to 11D may be directly coupled to a digital processing system, such that on board a nacelle, or may be a component of a digital application on a separate device, such as a smartphone, table, smart TV, and the like. As such, various embodiments of the athlete feedback systems and methods as herein described may comprise various digital communication means known in the art for sending and receiving data over any one or more of various means known in the art. Similarly, such data, and any information related to modeling, user tracking, computational algorithms, and the like, may be stored and/or accessed by a device to enable the embodiments herein described. As such, performance metrics, such as an Aerosmart score or athlete efficiency, may be tracked, stored, or shared between devices. For example, users may access or view performance metrics from previous activities, or may compare with those of other users. As such, the methods and systems herein described may serve, in some embodiments, as an avenue of competition between users that measures, for instance, normalised rider efficiency, rather than the more conventional “fastest time” to execute a particular distance or race.

Implementation of the various embodiments may be executed via a variety of computational methods and algorithms. For instance, various sensor systems may be employed to identify, for example, a body position, angle, or range thereof, that may be classified using computational models or machine learning algorithms as a given body position. For instance, and in accordance with various embodiments, a cycling position may be classified as one in which a torso angles falls within a designated range. That position may then have associated therewith one or more power-duration curves and CdA values which are used to determine an output efficiency in consideration of measured aerodynamics in real time.

Upon analysis, algorithms may employ lookup tables, stored, models, and the like for obtaining relevant parameters for further modeling and computation. Similarly, the skilled artisan will appreciate that data may be acquired over an appropriate duration of time in order to obtain reliable parameters, or to achieve a designated level of confidence to characterise a current or target body position, power output, velocity, and the like. Similarly, logical regression may be employed to determine, for instance, a probability of being in a particular body position based on, for instance, an amount of time an athlete spends is a particular angular range. Determination of body positions, either current or targeted for a given set of environmental parameters, may be based on, for instance, normal distributions of random variables, and may, in various embodiments, be based on specifically acquired data for a particular athlete, or from aggregate and/or crowd-sourced athlete data.

Further embodiments relate to modeling and computational structures that follow a decision tree framework. Such embodiments may, for instance, mitigate issues related to rates of failure in body position determination/recommendation, demand of computational resources and/or calculation speed, and the like. Similarly, Random Forest-based approaches may enable aggregate solutions from simpler decision tree computations and classifications.

In accordance with various embodiments, various power-duration fitting methods and statistical modeling may be employed to assist in decision-making of optimal or preferred body positions. For instance, Gaussian Regression Analysis may be used with low-to-moderate sample sizes while providing flexibility for the addition of various parameters and allowing for the calculation of confidence intervals based on “distance” (e.g. deviation) from previously acquired or processed data. Alternatively, or additionally, Cluster Analysis may be used in the processing of training sets or historical data to group multi-variate athlete data into subgroups for improved analysis of body position and/or duration efforts for one or more athletes performing an activity. Furthermore, and in accordance with various embodiments, models and/or parameters may be continuously or periodically updated based on acquired data to improve decision-making.

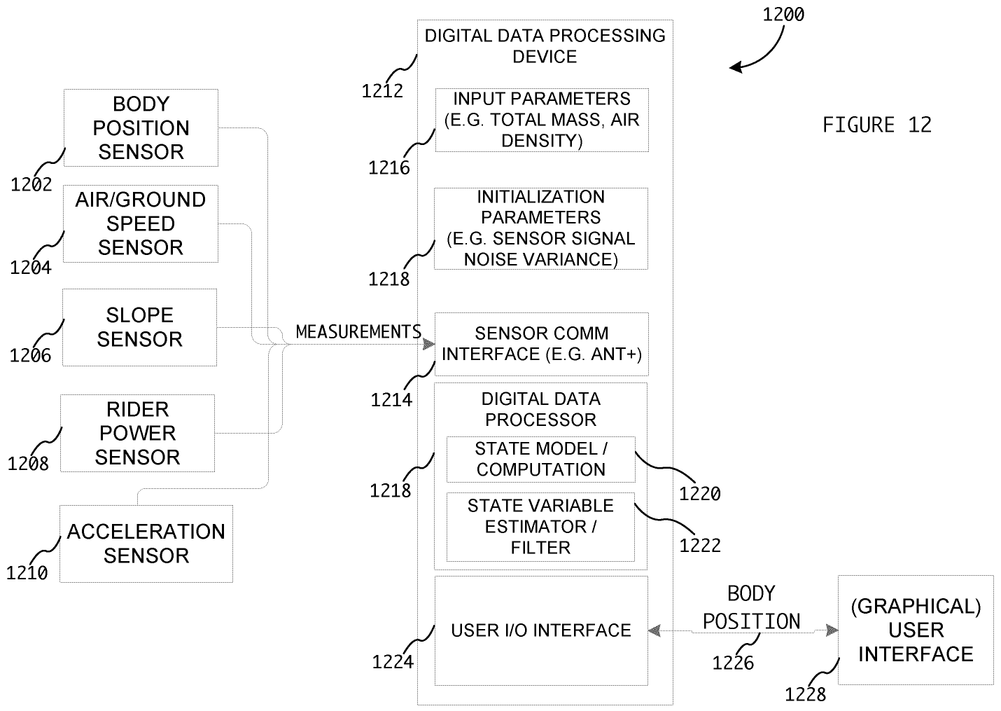

With reference to FIG. 12, a schematic diagram of an athlete body position optimisation and feedback system, generally referred to using the numeral 1200, will now be illustratively described. In the illustrated embodiment, and referring for simplicity to the application of cycling, the system comprises or is adapted to communicatively interface with one or more sensors, and generally a number of such sensors, that can be used as input variables to the cyclist's dynamic state model to estimate and/or compute various unknown variables such as an aerodynamic drag value or indication. In the illustrated embodiment, the system is configured to operate on readings acquired via one or more of an air (or wind) speed sensor and ground speed sensor, collectively referred to as element 1204, a slope sensor 1206 (e.g. inclinometer(s), gyroscope(s), accelerometer(s), etc.), a rider power sensor 1208 and/or an acceleration sensor 1210. Various embodiments further relate to the input of data related to a current athlete body position, such a hip or torso angle of a cyclist, which may be used to infer, for instance, a frontal area, current body positions, associated rider output and aerodynamic drag parameters.

Data signals and/or values from each of these sensors are continuously or discretely (e.g. at a set data transfer rate) transferred to a digital data processing device 1212 operable to process such signals. While device 1212 is illustrated as a distinct device, it will be appreciated that various sensors may be integrally formed or associated with the device 1212 in a common form factor, as can be provided as a distinct device operatively communicating with one or more external (e.g. proprietary or third party) sensors. Accordingly, the device 1212 will include one or more sensor communication interfaces 1214 to interface with each of the internal and/or external sensors. Different communication protocols may naturally be invoked to implement this interface, as can different protocols be used for different sensors depending on the nature thereof, whether sensors are integrated or external, wired or wireless, etc. In the illustrated example, the device 1212 may take the form of or include a microcontroller that is specifically programmed to interface with each sensor wirelessly using an ANT+ or like protocol, common for athletic/physiologic monitoring sensor communications. Other communication protocols may also or alternatively be considered, such as based on Zigbee, Bluetooth to name a few.

In one embodiment, the digital data processing device 1212 may be configured to receive and/or store one or more input parameters 1216 useful in subsequent computations, such as for example, but not limited to, the total mass of the rider and vehicle, air density, etc. While such input values may be useful, the systems and methods as described herein may also be configured to estimate any of these values which, in some embodiments, may provide for greater accuracy, for example, where a rider gradually consumes water from a mounted water bottle that will inherently reduce the overall weight, and like considerations. The system further initializes a number of initialization parameters 1218, such as in the following example, but not limited to, initial sensor and estimated value error ranges (the former generally derived from manufacturer specifications), directional tracking boundaries useful in mitigating potential windup issues in tracked/estimated values, and identification of which parameters are to be influenced by actual sensor readings as opposed to pure estimated values.

Based on the initialized and input parameters, and the input of sensor data, a digital data processor 1218 may be operated, based on a stored state model and computational process 1220, to act on these values to filter and estimate various state variables 1222 of interest. For instance, a state model may comprise a set of power duration curves corresponding to various athlete body positions, as illustratively depicted in FIG. 6, with associated aerodynamic force models based on predicted or measured frontal areas corresponding to respective body positions. Processor outputs may then be directed to an input/output interface 1224 to provide an output indication as to an unknown state variable of interest, such as an optimal or preferred body position 1226 in consideration of aerodynamics and expected output power, and/or an index or indicia corresponding thereto, for example. Process outputs may be stored and managed locally for further processing, output on a local (graphical) user interface, or again relayed via wired or wireless communications to an external or third-party interface 1228. Various embodiments further relate to the output and/or display of various processed values, such as an Aerosmart score as described above to provide real-time feedback on an athlete efficiency with respect to body position in consideration of aerodynamic forces, output powers, velocities, and the like.

In one embodiment, the digital data processor 1218 is configured to implement, for instance, a filter process at 1222 (e.g. an adapted extended Kalman Filter process), which may be configured to address the particular conditions at hand, namely to integrate known measurements over time, each containing statistical noise and/or other inaccuracies, and to produce estimates for unknown variables such as CdA and/or Crr by estimating a joint probability distribution over the variables for each timeframe. The processor may further be configured to access a digital database comprising historical and/or predicted performance parameters, such as power duration curves for a particular athlete or aggregate statistics related thereto from a plurality of cyclists.

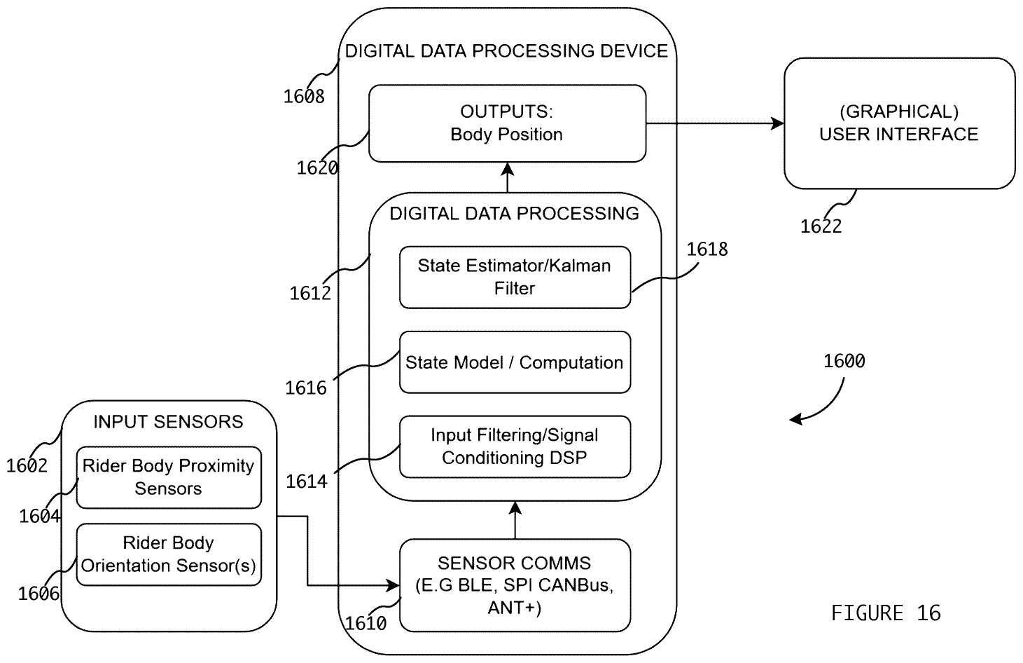

With reference to FIG. 16, a schematic diagram of an athlete body position optimization and feedback system, generally referred to using the numeral 1600, will now be illustratively described. In the illustrated embodiment, the system 1600 may comprise, or be adapted to communicatively interface with, one or more input sensors 1602 related to an athlete's body position, non-limiting examples of which may include a rider body proximity sensor 1604, a rider body orientation sensor 1606, or the like. Data from input sensors 1602 may be communicated to a digital data processing device 1608 via one or more sensor communication interfaces 1610 (e.g. a BLE, SPI CANBus, ANT+, or like interface) associated therewith for processing with a digital data processor 1612. The processor 1612 may in turn comprise modules or processes for, for instance, input data filtering, signal conditioning, or digital signal processing 1614, and may additionally or alternatively comprise various state models 1616, state estimators 1618, and/or filters 1618 for processing input data. The processor 1612 may further provide output 1620 related to, for instance, athlete body position. A signal related to the athletic body position may then be output from the processing device 1608 for display to an athlete or coach via, for instance, a graphical user interface 1622.

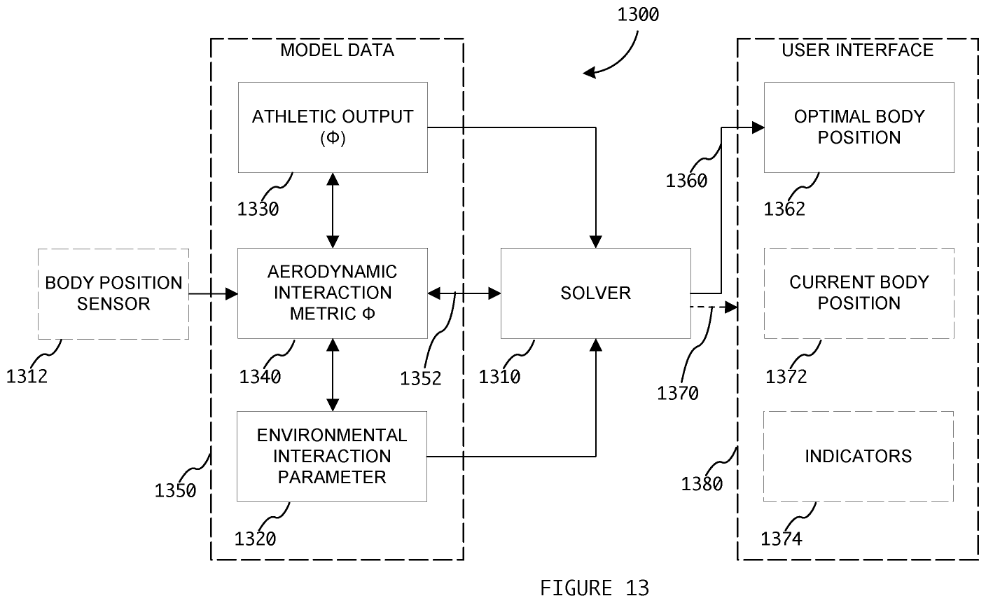

With reference to FIG. 13, a schematic diagram illustrating an exemplary process for determining an optimal or improved athletic body position for an athlete performing an activity will now be described. In the illustrated embodiment, the process may employ a solver 1310, such as a computer readable medium or the like with digital instructions thereon executable to process data from one or more data sources to determine an optimal or preferred body position 1360 of an athlete for a designated or predetermined target output. For instance, the solver 1310 may receive as input or otherwise determine a target cyclist velocity or target rider efficiency, and process received input so to determine a body position or athlete action that would result in a target outcome (e.g. an improved cycle speed) with the least amount of rider effort. Conversely, using the example of the cyclist and in accordance with another embodiment, the solver 1310 may determine a maximised speed that the cyclist may achieve for a designated rider effort level. Such target outputs may relate to, for instance, a current rider effort level or speed as determined by one or more sensors, or may be designated based on a particular athletic goal.

In this exemplary embodiment, the solver 1310 may receive an environmental interaction parameter 1320, such as a wind or air speed as measured during performance of the athletic activity (e.g. cycling). For instance, an air speed sensor worn by a cyclist (or coupled with a bicycle) may measure and communicate to the solver 1310 an air speed and/or direction relative to the cyclist. The environmental interaction parameter 1320 such as air speed may, in accordance with some embodiments, interact with a cyclist as a function of her body position. For instance, and as described above, an athlete may have a body position resulting in a cross-sectional body area A that interacts with air flow to produce a particular aerodynamic drag. In at least one embodiment, a process 1300, or system or device employing the same, may relate to a solver 1310 receiving as input both an environmental interaction parameter 1320 and an aerodynamic interaction metric 1340 related to an athlete's body position for processing. In other embodiments, an aerodynamic interaction metric 1340 and environmental interaction parameter 1330 may be preprocessed, for instance to provide the solver 1310 with various metrics related to aerodynamics, such as a CdA.

As discussed above, an athlete's body position may further be related to an athletic output 1330, such as a power output or output efficiency while performing an activity. For example, a cyclist in a particular body position may have a frontal area to produce a particular aerodynamic drag, while that body position further relates to a particular power-duration output curve. As such, various embodiments relate to an athletic output 1330 that may be input to a solver 1310 as a function of an aerodynamic metric Φ 1320.

The skilled artisan will appreciate that a solver 1310 may receive as input one or more athletic output functions 1330 that have been pre-selected based on a measured or inferred aerodynamic interaction metric 1340, or may receive one or more aerodynamic interaction metrics 1340 while also accessing a database of various athletic output functions 1320 and/or environmental interaction parameter functions 1320 to perform calculations. Similarly, any one or more of an environmental interaction parameter 1320, aerodynamic interaction metric 1340, and athletic output 1330 may be calculated or inferred based on one or more models 1330 or algorithms prior to input to the solver 1310. In some embodiments, a solver 1310 may access any combination(s) of the parameters discussed above in additional to any necessary or relevant models 1350.

For instance, and in accordance with various embodiments, an aerodynamic interaction metric 1340 may be measured or inferred from a body position sensor 1312 by comparing sensor data with a known model for a particular athlete or set of athletes. For example, one or more sensors on a cyclist may be used to infer an average hip flexion/angle, which may in turn be used to determine an estimated torso angle via, for instance a best fit to known relationships between these parameters. An estimated torso angle may then be input into one or more algorithms or models for estimating a frontal area to determine an aerodynamic drag, as well as compared with athletic output models to produce an expected athletic output for the estimated body position.

As discussed above, models 1320 may comprise various forms known in the art, such as lookup tables, or learned and/or continuously updated relationships. For example, a particular hip angle may translate readily to a body frontal area, or a model may be a function fit to rider output data previously acquired from aggregate statistics of multiple riders, or from previous performances of the same rider. Further, a model 1350 may further be employed to extract one or more parameters. For instance, an environmental interaction parameter 1320, such as a CdA, may be used to extract an aerodynamic interaction metric 1340, such as body area A. This may in turn may be used to determine an athletic output 1330 for a corresponding body position. Conversely, a measured athletic output 1320, such as a power-duration curve, may be used to infer a body position, which may in turn be correlated to a frontal area, and therefore an aerodynamic drag in a particular environment. Naturally, it is understood that any parameters necessary for performing calculations or extracting data, such as body weight, shoulder angle, height, and the like, may be accessed or input into any data model 1350 or solver 1310.

In accordance with various embodiments, a solver 1310 may further be operable to iteratively perform calculations to determine an optimal or improved body position for any input variable. For instance, an in accordance with one embodiment, a body position sensor 1312 may be employed to determine a current body position, and associated Φ 1340 for input to a solver 1310 for solving an energy balance equation. The solver 1310 may request or otherwise obtain as input a different Φ, as well as any associated models 1350 relating to athletic output 1330 and/or environmental interaction parameters 1320, to iteratively determine an improved body position.

In the illustrative example of FIG. 13, the solver 1310 may perform calculations and output a signal 1360 corresponding to an estimated improved or optimal body position 1362. The signal 1360 may, for instance, be stored for future processing, such as if a cyclist or coach thereof wishes to analyse performance of an athletic activity between sessions. Alternatively, a feedback signal 1370 corresponding to an optimal body position 1362 may be output in real- or near-real time to indicate to an athlete or coach, for instance via a user interface 1380, what a preferred or optimal position may be, given various measured, calculated, or estimated parameters during performance of the activity. For instance, a solver 1310 may receive as input a measured air speed 1320 and an estimated athletic output for a range of possible body positions (and therefore aerodynamic interaction metrics 1340). It may then compute an optimal body position 1362 from a set of possible body positions for a particular cyclist based on models generated from historical data of that athlete. The optimal position 1362 may be communicated via a feedback signal 1370 to the athlete via the interface 1380 to indicate that the rider should, for instance, “Stand Up”, or “Assume the Aerodynamic position”.

In yet other embodiments, a feedback signal 1370 may comprise information related to an athlete's current body position 1372, as determined from, for instance, a body position sensor 1312. For example, a feedback signal 1370 may indicate a preferred shift in the current body position 1372 to achieve the preferred position 1362 (e.g. “Get Down!”, “You are in the Aerodynamic Position, you should have your hands on the top bar”, etc.), which may be displayed on an interface 1380. An interface 1380 may, in some embodiments, further communicate via indicators 1374 any alternative information, such as an Aerosmart score, a wind speed, estimated lost time, current efficiency for measured environmental conditions, another user's or users' Aerosmart score, and the like.

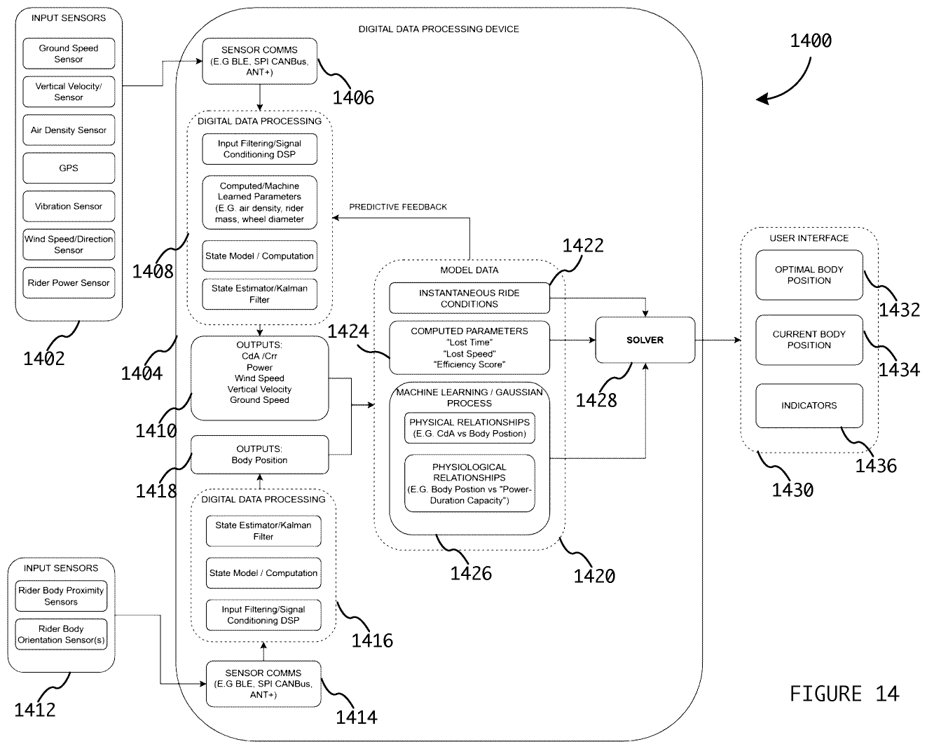

FIG. 14 schematically illustrates, in accordance with at least one embodiment, another exemplary process, generally referred to with the numeral 1400, for indicating an optimal or improved athletic body position for an athlete performing an activity. In this example, one or more aerodynamic, environmental, or like input sensors 1402, non-limiting examples of which may include a ground speed sensor, a vertical velocity sensor, an air density sensor, a GPS, a vibration sensor, a wind speed sensor, a wind direction sensor, a rider power sensor, or the like, may communicate sensor data to a digital data processing device 1404 via one or more sensor communication interfaces 1406 associated therewith for processing with a digital data processor 1408. The processor 1408 may in turn comprise modules or processes for, for instance, input data filtering, signal conditioning, or digital signal processing, and may additionally or alternatively comprise computed and/or machine-learned parameters related to, for instance, air density, rider mass, wheel diameter, or the like. The processor 1408 may, in some embodiments, comprise various state models, state estimators, and/or filters for processing input data. The processor 1408 may provide output 1410 related to, for instance, CdA, Crr, power, wind speed, vertical velocity, ground speed, or the like.

The process 1400 may further comprise acquiring sensor data via one or more additional sensors 1412 related to an athlete condition, non-limiting examples of which may include rider body proximity and/or orientation sensors. Sensor data may be communicated via one or more respective communication interfaces 1414 to the processing device 1404 for processing using digital data processing resources 1416 to output 1418 data related to, for instance, a rider's body position (e.g. hip or torso angle). In this example, processor outputs 1410 and 1418 are input as data into models 1420 associated with the processing device 1404. Models 1420 may calculate or otherwise infer, for instance, a rider's current condition(s) 1422, and/or computed parameters 1424 related to athletic performance (e.g. “lost time”, “lost speed”, efficiency or an “efficiency score”, or the like). Various other athletic models 1426 may further relate to the determination of various other athletic parameters based on model input data, such as a physical relationship between an athlete's CdA and body position, and/or one or more physiological relationships, such as an athlete's power-duration capacity for one or more body positions. In accordance with some embodiments, data generated from models 1420 may be accessed by digital data processors 1408 and/or 1416 for, for instance, predictive feedback processes.

In this exemplary embodiment, data generated from athletic models 1420 may be input into a digital solver 1428 associated with the processing device 1404 to output a signal representative of relevant or desired information to be displayed by a user interface 1430 to an athlete or coach. For instance, a graphical user interface 1430, such as one that may be displayed by a smartphone application, may be mounted on the handlebars of a bicycle to display to a cyclist an optimal body position 1432 for the current cycling conditions, as well as the cyclists current body position 1434, and any additional indicators 1436 of interest (e.g. an Aerosmart score, an efficiency, etc.).

While the present disclosure describes various embodiments for illustrative purposes, such description is not intended to be limited to such embodiments. On the contrary, the applicant's teachings described and illustrated herein encompass various alternatives, modifications, and equivalents, without departing from the embodiments, the general scope of which is defined in the appended claims. Except to the extent necessary or inherent in the processes themselves, no particular order to steps or stages of methods or processes described in this disclosure is intended or implied. In many cases the order of process steps may be varied without changing the purpose, effect, or import of the methods described.

Information as herein shown and described in detail is fully capable of attaining the above-described object of the present disclosure, the presently preferred embodiment of the present disclosure, and is, thus, representative of the subject matter which is broadly contemplated by the present disclosure. The scope of the present disclosure fully encompasses other embodiments which may become apparent to those skilled in the art, and is to be limited, accordingly, by nothing other than the appended claims, wherein any reference to an element being made in the singular is not intended to mean “one and only one” unless explicitly so stated, but rather “one or more.” All structural and functional equivalents to the elements of the above-described preferred embodiment and additional embodiments as regarded by those of ordinary skill in the art are hereby expressly incorporated by reference and are intended to be encompassed by the present claims. Moreover, no requirement exists for a system or method to address each and every problem sought to be resolved by the present disclosure, for such to be encompassed by the present claims. Furthermore, no element, component, or method step in the present disclosure is intended to be dedicated to the public regardless of whether the element, component, or method step is explicitly recited in the claims. However, that various changes and modifications in form, material, work-piece, and fabrication material detail may be made, without departing from the spirit and scope of the present disclosure, as set forth in the appended claims, as may be apparent to those of ordinary skill in the art, are also to encompassed by the disclosure.

1. A computer-readable medium having digital instructions stored thereon and executable by one or more digital processors to automatically determine an optimal athletic body position for an athlete performing an activity, the instructions executable to:
access a digital dataset comprising a plurality of respective athletic body positions, each having associated therewith a respective aerodynamic interaction metric and a respective athletic output;
receive as input an environmental interaction parameter;
compute, at least in part using said environmental interaction parameter and said digital dataset, the optimal athletic body position for a designated athletic output; and
output a signal corresponding to the optimal athletic body position.

2. The computer-readable medium of claim 1, wherein the instructions are further executable to:
receive as input a current body position metric indicative of a current athletic body position during performance of the activity;
classify, based on said current body position metric, one of said respective athletic body positions as said current athletic body position; and
compare said current athletic body position with the optimal athletic body position;
wherein said signal corresponding to the optimal athletic body position further comprises information related to said current athletic body position,
wherein the instructions are executable during performance of the activity.

3. (canceled)

4. The computer-readable medium of claim 2, wherein the instructions are further executable to quantify an athletic output efficiency based at least in part on said current athletic body position and the optimal athletic body position, and wherein said signal further comprises data related to said athletic output efficiency.

5. The computer-readable medium of claim 4, wherein the instructions are further executable to track said athletic output efficiency over a designated duration of the physical activity so to provide an accumulated output efficiency.

6. (canceled)

7. The computer-readable medium of claim 1, wherein said respective athletic output is related to one or more of an energy output, a power output, a metabolic cost, or a power duration curve.

8. The computer-readable medium of claim 1, wherein said designated athletic output corresponds to one or more of a designated speed, a designated power, a designated efficiency, or a designated athletic output as a function of metabolic cost.

9. The computer-readable medium of claim 1, wherein a designated relationship between said plurality of respective athletic body positions, said respective aerodynamic interaction metrics, and said respective athletic outputs is predetermined from data acquired from prior performance of the athletic activity by at least one of the athlete or multiple athletes.

10. (canceled)

11. A system for automatically determining an optimal athletic body position for an athlete performing an activity, the system comprising:
a digital data processor;
a user interface; and
a computer-readable medium having digital instructions stored thereon and executable by said digital processor to:
access a digital dataset comprising a plurality of respective athletic body positions, each having associated therewith a respective aerodynamic interaction metric and a respective athletic output;
receive as input an environmental interaction parameter;
compute, at least in part using said environmental interaction parameter and said digital dataset, the optimal athletic body position for a target athletic output; and
display via said user interface a signal corresponding to the optimal athletic body position.

12. The system of claim 11, wherein said instructions are further executable to:
receive as input a current body position metric indicative of a current athletic body position during performance of the activity;
classify, based on said current body position metric, one of said respective athletic body positions as said current athletic body position; and
compare said current athletic body position with the optimal athletic body position;
wherein said signal corresponding to the optimal athletic body position further comprises information related to said current athletic body position,
wherein the instructions are executed during performance of the activity and operable to provide real-time feedback related to the athlete.

13. (canceled)

14. The system of claim 12, wherein the instructions are further executable to quantify an athletic output efficiency based at least in part on said current athletic body position and the optimal athletic body position, and wherein said signal further comprises data related to said athletic output efficiency.

15. The system of claim 14, wherein the instructions are further executable to track said athletic output efficiency over a designated duration of the physical activity so to provide an accumulated output efficiency.

16. The system of claim 11, wherein the instructions are further executable to communicate said signal to a third-party digital application using an internet protocol or a wired connection.

17. The system of claim 11, wherein said respective athletic output is related to one or more of an energy output, a power output, a metabolic cost, or a power duration curve.

18. The system of claim 11, wherein said designated athletic output corresponds to one or more of a designated speed, a designated power, a designated efficiency, or a designated athletic output as a function of metabolic cost.

19. The system of claim 11, wherein a designated relationship between said plurality of respective athletic body positions, said respective aerodynamic interaction metrics, and said respective athletic outputs is predetermined from data acquired from prior performance of the athletic activity by at least one of the athlete or multiple athletes.

20. (canceled)

21. A method of automatically determining an optimal athletic position for an athlete performing an activity, the method to be implemented by one or more digital data processors and comprising:
accessing a digital dataset comprising a plurality of respective athletic body positions, each having associated therewith a respective aerodynamic interaction metric and a respective athletic output;
receiving as input an environmental interaction parameter;
computing, at least in part using said environmental interaction parameter and said digital dataset, the optimal athletic body position for a target athletic output; and
outputting a signal corresponding to the optimal athletic body position.

22. The method of claim 21, further comprising:
receiving as input a current body position metric indicative of a current athletic body position during performance of the activity;
classifying, based on said current body position metric, one of said respective athletic body positions as said current athletic body position; and
comparing said current athletic body position with the optimal athletic body position;
wherein said signal corresponding to the optimal athletic body position further comprises information related to said current athletic body position,
wherein the method further comprises displaying said signal in real-time to the athlete.

23. (canceled)

24. The method of claim 22, further comprising:
quantifying an athletic output efficiency based at least in part on said current athletic body position and the optimal athletic body position, and wherein said signal further comprises data related to said athletic output efficiency.

25. The method of claim 24, further comprising:
tracking said athletic output efficiency over a designated duration of the physical activity so to provide an accumulated output efficiency.

26. (canceled)

27. The method of claim 21, further comprising:
generating said dataset from data acquired from prior performance of the athletic activity by at least one of the athlete or multiple athletes.

28. (canceled)
